(19) United States
(12) Patent Application Publication
(10) Pub. No.: US 2021/0087289 A1
(43) Pub. Date: Mar. 25, 2021

(54) THERAPEUTIC ANTIBODY AND USES THEREOF

(71) Applicant: Abmart Shanghai Co., LTD, Shanghai (CN)

(72) Inventors: Xun Meng, Shanghai (CN); Jian-Xin Bao, Shanghai (CN); Bing Hou, Shanghai (CN)

(21) Appl. No.: 16/996,949

(22) Filed: Aug. 19, 2020

Related U.S. Application Data
(63) Continuation of application No. PCT/CN2018/076958, filed on Feb. 22, 2018.

(51) Int. Cl. C07K 16/28; A61K 47/68; C07K 16/30; A61P 35/00

(52) U.S. Cl. CPC C07K 16/2884; A61K 47/6803; A61P 35/00; C07K 16/30; A61K 47/6849

(57) ABSTRACT

The invention described herein provides antibodies or antigen-binding fragment thereof specific for an epitope within the variant exon v6 or v9 of the CD44 gene (CD44v6 or CD44v9), antibody-drug-conjugate (ADC) thereof, and other derivative comprising the antibodies or antigen-binding fragment thereof. The invention also provides nucleic acid molecules encoding the same, and methods of making the same. The invention further provides pharmaceutical compositions comprising the same, and the use of the same in treating diseases or in the manufacture of a medicament for the treatment of the diseases, such as cancer.

### REFERENCE TO RELATED APPLICATION

This application is a U.S. continuation application of International Patent Application No. PCT/CN2018/076958, filed on Feb. 22, 2018, published as WO 2019/161528, the entire content of which, including all drawings and sequences are incorporated herein by reference.

### REFERENCE TO SEQUENCE LISTING

This application contains a Sequence Listing in computer readable form, which has been submitted electronically via EFS-web in ASCII format. Said ASCII copy, created on Nov. 2, 2020, is named 128763_00102_Seq_Listing.txt, and is 12,822 bytes in size. The computer readable form of the sequence listing are part of the specification or are otherwise incorporated herein by reference.

### BACKGROUND OF THE INVENTION

CD44 is a family of transmembrane glycoproteins involved in homotypic cell, cell-matrix, and cell-cytoskeletal interaction. The extracellular domain of CD44 binds numerous matrix substituents: hyaluronic acid, ezrin, radixin, moesin and merlin, heparin-affinity growth factors, vascular endothelial growth factor, p185HER2, epidermal growth factor, and hepatocyte growth factor. Its intracellular domain binds the cytoskeletal substituent ankyrin, thus determining cell and tissue architectural form (Bourguignon et al., 1998, Front. Biosci. 3:D637-649; Welch et al., 1995, J. Cell. Physiol. 164:605-612).

The CD44 gene, which maps to chromosome 11, contains 20 exons spanning 60 kb, and can be subdivided into 5 structural domains. Ten of the 20 exons, exons 1-5 and 16-20, constitute the standard form of CD44 (CD44s or CD44std). While this smallest CD44 isoform (CD44s) is ubiquitously expressed in different tissues including epithelial cells, certain CD44 splice variants (CD44v, CD44var) are expressed only on a subset of epithelial cells.

The CD44 variants are generated by alternative splicing at the messenger RNA (mRNA) level, in a way that the sequences of ten exons (v1-v10) in the extracellular portion of the protein are completely excised in CD44s but can appear in the bigger variants in different combinations (Screaton et al., 1992; Tölg et al., 1993; Hofmann et al., 1991). The variants differ in that different amino acid sequences are inserted at a certain site of the extracellular part of the protein. Theoretically, there are over 1000 potential peptide domain combinations for CD44 variant (CD44v) isoforms. Inclusion of all variant exons would yield a protein of molecular weight 230 kD, but most variant isoforms are less than 120 kD. Longer, variant isoforms (CD44v1-10) include one or more of exons 6-15 spliced in, although in humans, exon 6 (v1) is not expressed.

Some splice variants are expressed by normal epithelial cells in a tissue-specific fashion. For example, CD44v10 is expressed by normal lymphocytes (Okamoto et al., 1998,

J. Natl. Cancer Inst. 90:307-315).

Recently, however, it has been shown that the expression of certain CD44 variants is necessary and sufficient for causing so-called spontaneous metastatic behaviour of a non-metastasizing rat pancreatic adenocarcinoma cell line as well as a non-metastasizing rat fibrosarcoma cell line (Günthert et al., 1991). Such variants can be detected in various human tumor cells as well as in human tumor tissue.

For example, Okamoto et al. (Okamoto et al., 2002, Am. J. Pathol. 160:441-447; Okamoto et al., 2001, J. Cell. Biol. 155:755-762; Murakami et al., 2003, Oncogene 22:1511-1516) have shown that, in several human tumors (exclusive of prostate cancer), cleavage products of 25-30 kD are detectable by Western blot using antibody against the cytoplasmic portion of CD44. The soluble portion of CD44 has been detected in serum as a 100-160-kD fragment using anti-CD44v monoclonal antibodies to extracellular portions of the molecule (Gansauge et al., 1997, Cancer 80:1733-1739). Western blot detection of CD44 isoforms shed into the circulation may serve as a diagnostic or prognostic test for malignancy (Taylor et al., 1996, J. Soc. Gynecol. Invest. 3:289-294). An enzyme-linked serum immunoassay (ELISA) may then be developed for sensitive, easier detection of the proteins. Amplification in CD44v6 is noted with metastatic phenotype of pancreatic cancer (Rall and Rustgi, 1995, Cancer Res. 55:1831-1835) and increasing grade of breast cancer (Woodman et al., 1996, Am. J. Pathol. 149:1519-1530; Bourguignon et al., 1999, Cell Motil. Cytoskeleton 43:269-287). Inclusion of single or contiguous variant exons has been described by RT-PCR and sequencing in many benign and cancer tissues (Okamoto et al., 1998, J. Natl. Cancer Inst. 90:307-315; Okamoto et al., 2002, Am. J. Pathol. 160:441-447; Rall and Rustgi, 1995, Cancer Res. 55:1831-1835; Woodman, et al., 1996, Am. J. Pathol. 149:1519-1530; Bourguignon et al., 1999, Cell Motil. Cytoskeleton 43:269-287; Roca et al., 1998, Am. J. Pathol. 153:183-190; Franzmann et al., 2001, Otolaryngol. Head Neck Surg. 124:426-432; Terpe et al., 1996, Am. J. Pathol. 148:453-463; Christ et al., 2001, J. Leukoc. Biol. 69:343-352; Mortegani et al., 1999, Am. J. Pathol. 154:291-300; Miyake et al., 1998, Int. J. Cancer. 18:560-564; Yamaguchi et al., 1996, J. Clin. Oncol. 14:1122-1127).

During metastasis, tumor cells detach from the primary site, migrate into the extracellular matrix, and invade blood and lymph vessels. Tumor outgrowth at the metastatic site requires attachment to the new extracellular matrix through adhesion proteins such as CD44. This is consistent with the fact that many cancers have deregulated CD44 mRNA splicing, leading to expression of novel CD44 variant isoforms that may play a role in metastasis.

Indeed, the expression of CD44 variants in the course of colorectal carcinogenesis has recently been investigated (Heider et al., 1993a). The expression of CD44 variants is absent in normal human colon epithelium, and only a weak expression is detectable in the proliferating cells of the crypts. In later stages of the tumor progression, e.g., in adenocarcinomas, all malignancies express variants of CD44. Tissue expression of variant CD44 on a high level has also been shown in aggressive Non-Hodgkin lymphomas (Koopman et al., 1993).

Exon v6 appears to play a special role especially in the course of metastatic spread (Rudy et al., 1993). In an animal model, antibodies against v6 specific epitopes could prevent the settlement of metastatic cells and the growth of metastases (Seiter et al., 1993). In colon carcinomas, v6 expression correlates with tumor progression (Wielenga et al., 1993). In gastric carcinomas, v6 expression is an important diagnostic marker to distinguish tumors of the intestinal type from those of the diffuse type (Heider et al., 1993b). In the latter two publications, v6 expression has been determined using antibodies against v6 specific epitopes.

As CD44v6 has been shown to be a tumor-associated antigen with a favorable expression pattern in human tumors and normal tissues (Heider et al., 1995; Heider et al., 1996), it has been subject to antibody-based diagnostic and therapeutic approaches, (Heider et al., 1996; WO 95/33771; WO 97/21104).

On the other hand, abnormal expression of CD44v9 has been associated with gastric cancer, colon cancer, breast cancer, lung cancer, and head and neck squamous cell carcinoma (US20170137810A1). Both CD44v6 and CD44v9 were previously demonstrated to be over-expressed in colon cancer (Wielenga et al., Am. J. Pathol., 1999, 154: 515-523). CD44v9 has also been found to be over-expressed in gastric cancer (Ue et al., Co-expression of osteopontin and CD44v9 in gastric cancer. Int J Cancer 1998; 79:127-132).

CD44v9 positive cells demonstrate an enhanced ability to suppress the production of ROS, resulting in subsequent therapeutic resistance, recurrence, and metastasis of tumours (Ishimoto et al, 2011; Tsugawa et al, 2012; Yae et al, 2012). It has also been reported that CD44v9 was a cancer stem cell marker in a variety of tumor types (Aso et al., 2015).

One serious problem that arises when using non-human antibodies for applications in humans is that they quickly raise a human anti-non-human response that reduces the efficacy of the antibody in patients and impairs continued administration. To overcome that problem, concepts of “humanising” non-human antibodies have been developed in the art. In the first approach, humanization of non-human antibodies has been tried to achieve by constructing non-human/human chimeric antibodies, wherein the non-human variable regions are joined to human constant regions (Boulianne G. L., Hozumi N. and Shulman, M. J. (1984) Production of functional chimeric mouse/human antibody Nature 312: 643). The chimeric antibodies thus generated retain the binding specificity and affinity of the original non-human antibody.

However, chimeric antibodies, although significantly better than mouse antibodies, can still elicit an anti-chimeric response in humans (LoBuglio A. F., Wheeler R. H., Trang J., Haynes A., Rogers K., Harvey E. B., Sun L., Ghrayeb J. and Khazaeli M. B. (1989) Mouse/human chimeric monoclonal antibody in man: Kinetics and immune response. Proc. Natl. Acad. Sci. 86: 4220). This approach was later refined by further reducing the amount of non-human sequences by grafting the complementarity determining regions (CDRs) from the non-human variable regions to human variable regions and then joining these “reshaped human” variable regions to human constant regions (Riechmann L., Clark M., Waldmann H. and Winter G. (1988) Reshaping human antibodies for therapy. Nature 332: 323). CDR-grafted or reshaped human antibodies contain little or no protein sequences that can be identified as being derived from mouse antibodies. Although an antibody humanised by CDR-grafting may still be able to elicit some immune reactions, such as an anti-allotype or an anti-idiotypic response, as seen even with natural human antibodies, the CDR-grafted antibody will be significantly less immunogenic than a mouse antibody thus enabling a more prolonged treatment of patients.

However, it soon turned out that CDR-grafting alone did not always result in antibodies with sufficient binding affinity. CDR-grafted antibodies sometimes have relatively poor binding characteristics as compared to their parent non-human antibodies because, for example, more amino acids than those within the CDR's may be involved in antigen binding. As a consequence, CDR-grafted antibodies with poor binding affinity are not regarded to be useful in therapy. Therefore, attempts have been made to create antibodies which combine the low immunogenicity of CDR-grafted antibodies with the good binding characteristics of the non-human parent antibodies. The concept was developed that, in addition to CDR-grafting, one to several amino acids in the humanized framework region have to be retained as residues of rodent donor origin for retaining binding affinity (Queen et al, (1989) Proc. Natl. Acad. Sci. 86: 10029-10033).

Because of the high potential utility such antibodies could have in diagnosis and therapy, there is a need of antibodies with improved properties which are suitable for treatment of human disease, such as various cancers.

One problem underlying the present invention was to provide alternative CD44v6 or CD44v9 specific antibodies, preferably with better properties compared to the known CD44v6 or CD44v9 specific antibodies.

### SUMMARY OF THE INVENTION

One aspect of the invention provides an isolated monoclonal antibody, or an antigen-binding fragment thereof, specific for an isolated CD44v6 epitope, wherein said CD44v6 epitope comprises/consists essentially of SEQ ID NO: 19 (e.g., an epitope consisting of SEQ ID NO: 19 plus 1 or 2 residues on the N-terminus of SEQ ID NO: 19, SEQ ID NO: 19 plus 1 or 2 residues on the C-terminus of SEQ ID NO: 19, or SEQ ID NO: 19 plus 1 or 2 residues on both the N-terminus and the C-terminus of SEQ ID NO: 19), or consists of SEQ ID NO: 19; preferably, said antibody or antigen-binding fragment thereof is raised against said isolated CD44v6 epitope, or raised against a fusion protein or chemical conjugate thereof comprising said isolated CD44v6 epitope and a carrier protein (such as albumin, preferably BSA or ovalbumin, or keyhole limpet hemocyanin (KLH)).

In certain embodiments, the monoclonal antibody comprises: (1) a heavy chain variable region (HCVR), comprising a HCVR CDR1 sequence of SEQ ID NO: 1, a HCVR CDR2 sequence of SEQ ID NO: 2, and a HCVR CDR3 sequence of SEQ ID NO: 3; (2) a light chain variable region (LCVR), comprising a LCVR CDR1 sequence of SEQ ID NO: 10, a LCVR CDR2 sequence of SEQ ID NO: 11, and a LCVR CDR3 sequence of SEQ ID NO: 12.

In certain embodiments, the CD44v6 epitope is SEQ ID NO: 19.

In certain embodiments, the CD44v6 epitope consists essentially of SEQ ID NO: 19 (e.g., an epitope consisting of SEQ ID NO: 19 plus 1 or 2 residues on the N-terminus of SEQ ID NO: 19, SEQ ID NO: 19 plus 1 or 2 residues on the C-terminus of SEQ ID NO: 19, or SEQ ID NO: 19 plus 1 or 2 residues on both the N-terminus and the C-terminus of SEQ ID NO: 19).

In certain embodiments, the CD44v6 epitope is SEQ ID NO: 24 (HEGYRQTPKEDS).

In certain embodiments, (i) the HCVR further comprises one or more of SEQ ID NOs: 7-9; and/or, (ii) the LCVR further comprises one or more of SEQ ID NOs: 13-18.

In certain embodiments, the isolated monoclonal antibody or antigen-binding fragment thereof binds to said CD44v6 epitope, or a cell having said CD44v6 epitope, with a K_{D} of about 10 nM, about 5 nM, or about 2 nM or less.

In certain embodiments, the isolated monoclonal antibody or antigen-binding fragment thereof is a human-mouse chimeric antibody, a humanized antibody, a human antibody, a CDR-grafted antibody, or a resurfaced antibody.

In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In a related aspect, the invention provides an isolated monoclonal antibody, or an antigen-binding fragment thereof, wherein said isolated monoclonal antibody or antigen-binding fragment thereof binds to the same epitope of CD44v6 that is bound by a reference monoclonal antibody, or competes with said reference monoclonal antibody for binding to the same epitope of CD44v6, wherein said reference monoclonal antibody comprises: (1) a heavy chain variable region (HCVR), comprising a HCVR CDR1 sequence of SEQ ID NO: 1, a HCVR CDR2 sequence of SEQ ID NO: 2, and/or a HCVR CDR3 sequence of SEQ ID NO: 3; (2) a light chain variable region (LCVR), comprising a LCVR CDR1 sequence of SEQ ID NO: 10, a LCVR CDR2 sequence of SEQ ID NO: 11, and/or a LCVR CDR3 sequence of SEQ ID NO: 12.

Another aspect of the invention provides an isolated monoclonal antibody, or an antigen-binding fragment thereof, specific for an isolated CD44v9 epitope, wherein said CD44v9 epitope comprises/consists essentially of SEQ ID NO: 43 (e.g., an epitope consisting of SEQ ID NO: 43 plus 1 or 2 residues on the N-terminus of SEQ ID NO: 43, SEQ ID NO: 19 plus 1 or 2 residues on the C-terminus of SEQ ID NO: 43, or SEQ ID NO: 43 plus 1 or 2 residues on both the N-terminus and the C-terminus of SEQ ID NO: 43), or consists of SEQ ID NO: 43; preferably, said antibody or antigen-binding fragment thereof is raised against said isolated CD44v9 epitope, or raised against a fusion protein or chemical conjugate thereof comprising said isolated CD44v9 epitope and a carrier protein (such as albumin, preferably BSA or ovalbumin, or keyhole limpet hemocyanin (KLH)).

In certain embodiments, the monoclonal antibody comprises: (1) a heavy chain variable region (HCVR), comprising a HCVR CDR1 sequence of SEQ ID NO: 25, a HCVR CDR2 sequence of SEQ ID NO: 26, and a HCVR CDR3 sequence of SEQ ID NO: 27; (2) a light chain variable region (LCVR), comprising a LCVR CDR1 sequence of SEQ ID NO: 34, a LCVR CDR2 sequence of SEQ ID NO: 35, and a LCVR CDR3 sequence of SEQ ID NO: 36.

In certain embodiments, the CD44v9 epitope is SEQ ID NO: 43.

In certain embodiments, the CD44v9 epitope consists essentially of SEQ ID NO: 43 (e.g., an epitope consisting of SEQ ID NO: 43 plus 1 or 2 residues on the N-terminus of SEQ ID NO: 43, SEQ ID NO: 43 plus 1 or 2 residues on the C-terminus of SEQ ID NO: 43, or SEQ ID NO: 43 plus 1 or 2 residues on both the N-terminus and the C-terminus of SEQ ID NO: 43).

In certain embodiments, the CD44v9 epitope is SEQ ID NO: 44 (SHEGLEEDKDH).

In certain embodiments, (i) the HCVR further comprises one or more of SEQ ID NOs: 28-33; and/or, (ii) the LCVR further comprises one or more of SEQ ID NOs: 37-42.

In certain embodiments, the isolated monoclonal antibody or antigen-binding fragment thereof binds to said CD44v9 epitope, or a cell having said CD44v9 epitope, with a K_{D} of about 10 nM, about 5 nM, about 2 nM, about 1 nM or less.

In certain embodiments, the isolated monoclonal antibody or antigen-binding fragment thereof is a human-mouse chimeric antibody, a humanized antibody, a human antibody, a CDR-grafted antibody, or a resurfaced antibody.

In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In a related aspect, the invention provides an isolated monoclonal antibody, or an antigen-binding fragment thereof, wherein said isolated monoclonal antibody or antigen-binding fragment thereof binds to the same epitope of CD44v9 that is bound by a reference monoclonal antibody, or competes with said reference monoclonal antibody for binding to the same epitope of CD44v9, wherein said reference monoclonal antibody comprises: (1) a heavy chain variable region (HCVR), comprising a HCVR CDR1 sequence of SEQ ID NO: 25, a HCVR CDR2 sequence of SEQ ID NO: 26, and/or a HCVR CDR3 sequence of SEQ ID NO: 27; (2) a light chain variable region (LCVR), comprising a LCVR CDR1 sequence of SEQ ID NO: 34, a LCVR CDR2 sequence of SEQ ID NO: 35, and/or a LCVR CDR3 sequence of SEQ ID NO: 36.

Another aspect of the invention provides a polypeptide comprising the HCVR and/or the LCVR of any one of the subject anti-CD44v6 or anti-CD44v9 antibodies or antigen binding fragments thereof.

In certain embodiments, the polypeptide is a fusion protein (such as a chimeric antigen T cell receptor).

Another aspect of the invention provides a polynucleotide encoding any of the subject polypeptides.

Another aspect of the invention provides a vector comprising any of the subject polynucleotides.

In certain embodiments, the vector is an expression vector (e.g., a mammalian expression vector, a yeast expression vector, an insect expression vector, or a bacterial expression vector).

Another aspect of the invention provides a cell comprising any of the subject anti-CD44v6 or anti-CD44v9 antibody or antigen-binding fragment thereof, any of the subject polypeptide, any of the subject polynucleotide, or any of the subject vector.

In certain embodiments, the cell expresses any of the subject antibody or antigen-binding fragment thereof, or any of the subject polypeptide.

In certain embodiments, the cell is a BHK cell, a CHO cell, or a COS cell.

In certain embodiments, the cell comprises any of the subject anti-CD44v6 or anti-CD44v9antibody or antigen-binding fragment thereof, or any of the subject polypeptide, on the surface of the cell.

In certain embodiments, the cell is a T-cell bearing a chimeric antigen receptor (CAR-T cell) comprising any of the subject antibody or antigen-binding fragment thereof, or any of the subject polypeptide.

Another aspect of the invention provides an isolated CD44v6 epitope comprising/consisting essentially of SEQ ID NO: 19 (e.g., an epitope consisting of SEQ ID NO: 19 plus 1 or 2 residues on the N-terminus of SEQ ID NO: 19, SEQ ID NO: 19 plus 1 or 2 residues on the C-terminus of SEQ ID NO: 19, or SEQ ID NO: 19 plus 1 or 2 residues on both the N-terminus and the C-terminus of SEQ ID NO: 19), or consisting of SEQ ID NO: 19.

Another aspect of the invention provides a fusion protein or chemical conjugate comprising the isolated CD44v6 epitope of claim 28, and a carrier protein (such as albumin, preferably BSA or ovalbumin, or keyhole limpet hemocyanin (KLH)).

Another aspect of the invention provides an isolated CD44v9 epitope comprising/consisting essentially of SEQ ID NO: 43 (e.g., an epitope consisting of SEQ ID NO: 43 plus 1 or 2 residues on the N-terminus of SEQ ID NO: 43, SEQ ID NO: 43 plus 1 or 2 residues on the C-terminus of SEQ ID NO: 43, or SEQ ID NO: 43 plus 1 or 2 residues on both the N-terminus and the C-terminus of SEQ ID NO: 43), or consisting of SEQ ID NO: 43.

Another aspect of the invention provides a fusion protein or chemical conjugate comprising the isolated CD44v9 epitope of claim 28a, and a carrier protein (such as albumin, preferably BSA or ovalbumin, or keyhole limpet hemocyanin (KLH)).

Another aspect of the invention provides a method of producing any of the subject anti-CD44v6 or anti-CD44v9 antibody or antigen-binding fragment thereof, or any of the subject polypeptide, comprising: (a) culturing any of the subject cell; and, (b) isolating said antibody, antigen-binding fragment thereof, or polypeptide from said cultured cell.

In certain embodiments, the cell is a eukaryotic cell.

Another aspect of the invention provides an immunoconjugate (or antibody-drug conjugate or ADC) having the following formula: Ab-[-L-D]ₙ, wherein: Ab is any of the subject anti-CD44v6 or anti-CD44v9 antibody or antigen-binding fragment thereof, or any of the subject polypeptide thereof, that is covalently linked to one or more units of linker-drug moieties -[-L-D], wherein L is a linker and D is a cytotoxic drug; and, n is an integer from 1 to 20 (e.g., from 1-12); and wherein each linker-drug moiety may have the same or different linker L or cytotoxic drug D.

In certain embodiments, each linker-drug moiety -[-L-D] is covalently linked to Ab via a sidechain amino group of Lys.

In certain embodiments, each linker-drug moiety -[-L-D] is covalently linked to Ab via a sidechain thiol group of Cys.

In certain embodiments, each linker-drug moiety -[-L-D] is covalently linked to Ab via a site-specifically incorporated non-natural amino acid.

In certain embodiments, each linker L comprises a peptide unit.

In certain embodiments, the peptide unit comprises 2, 3, 4, 5, 6, 7, 8, 9, 10, 2-10, or 2-5 amino acid residues.

In certain embodiments, the linker L is non-cleavable by protease (e.g., cathepsin).

In certain embodiments, the linker L is a cleavable linker cleavable by protease (e.g., cathepsin), acidic environment, or redox state change.

In certain embodiments, the cytotoxic drug is a DNA intercalating agent, a microtubule binder, a topoisomerase I inhibitor, or a DNA minor groove binder.

In certain embodiments, the cytotoxic drug is auristatin class such as monomethyl auristatin E (MMAE) and MMAF, maytansine class such as DM-1, DM-3, DM-4, calicheamicin such as ozogamicin, SN-38, or PBD (pyrrolobenzodiazepin).

Another aspect of the invention provides a pharmaceutical composition comprising any of the subject anti-CD44v6 or anti-CD44v9 antibody or antigen-binding fragment thereof, or the polypeptide thereof, or the immunoconjugate thereof, and a pharmaceutically acceptable carrier or excipient.

Another aspect of the invention provides a method for inhibiting the growth of a cell expressing CD44v6, comprising contacting the cell with any of the subject anti-CD44v6 antibody or antigen-binding fragment thereof, or the subject polypeptide thereof, or the subject immunoconjugate thereof, or the subject pharmaceutical composition thereof.

In certain embodiments, the cell is a tumor cell.

In certain embodiments, the tumor cell is from a lung cancer (such as NSCLC).

In certain embodiments, the tumor cell is from colorectal cancer, breast cancer, head and neck cancer, ovarian cancer, bladder cancer, pancreatic cancer, or metastatic cancers of the brain.

Another aspect of the invention provides a method for inhibiting the growth of a cell expressing CD44v9, comprising contacting the cell with any of the subject anti-CD44v9 antibody or antigen-binding fragment thereof, or the polypeptide thereof, or the immunoconjugate thereof, or the pharmaceutical composition thereof.

In certain embodiments, the cell is a tumor cell.

In certain embodiments, the tumor cell is from a lung cancer (such as NSCLC).

In certain embodiments, the tumor cell is from colorectal cancer, breast cancer, liver, head and neck cancer, ovarian cancer, bladder cancer, pancreatic cancer, or metastatic cancers of the brain.

Another aspect of the invention provides a method for treating a subject having cancer, wherein cells of the cancer expresses CD44v6, the method comprising administering to said subject a therapeutically effective amount of an antagonist of CD44v6 comprising a CD44v6 antibody or an antigen-binding fragment thereof.

Another aspect of the invention provides a method for treating a cell-proliferative disorder in a subject, wherein cells of the cell-proliferative disorder expresses CD44v6, the method comprising administering to said subject a therapeutically effective amount of an antagonist of CD44v6 comprising a CD44v6 antibody or an antigen-binding fragment thereof.

In certain embodiments, the antagonist of CD44v6 comprises any of the subject anti-CD44v6 antibody or antigen-binding fragment thereof, or the subject polypeptide thereof, or the subject immunoconjugate thereof, or the subject pharmaceutical composition thereof.

In certain embodiments, the cancer is an epithelial carcinoma including breast, lung, liver, colorectal, head and neck, esophageal, pancreatic, ovarian, bladder, gastric, skin, endometrial, ovarian, testicular, esophageal, prostatic or renal origin; a bone and soft-tissue sarcoma such as osteosarcoma, chondrosarcoma, fibrosarcoma, malignant fibrous histiocytoma (MFH), leiomyosarcoma; a hematopoietic malignancy such as Hodgkin's lymphoma, non-Hodgkin's lymphoma, or leukemia; a neuroectodermal tumor such as peripheral nerve tumor, astrocytoma, or melanoma; or a mesotheliomas.

Another aspect of the invention provides a method for treating a subject having cancer, wherein cells of the cancer expresses CD44v9, the method comprising administering to said subject a therapeutically effective amount of an antagonist of CD44v9 comprising a CD44v9 antibody or an antigen-binding fragment thereof.

Another aspect of the invention provides a method for treating a cell-proliferative disorder in a subject, wherein cells of the cell-proliferative disorder expresses CD44v9, the method comprising administering to said subject a therapeutically effective amount of an antagonist of CD44v9 comprising a CD44v9 antibody or an antigen-binding fragment thereof.

In certain embodiments, the antagonist of CD44v9 comprises any of the subject anti-CD44v9 antibody or antigen-binding fragment thereof, or the polypeptide thereof, or the immunoconjugate thereof, or the pharmaceutical composition thereof.

In certain embodiments, the cancer is an epithelial carcinoma including breast, lung, liver, colorectal, head and neck, esophageal, pancreatic, ovarian, bladder, gastric, skin, endometrial, ovarian, testicular, esophageal, prostatic or renal origin; a bone and soft-tissue sarcoma such as osteosarcoma, chondrosarcoma, fibrosarcoma, malignant fibrous histiocytoma (MFH), leiomyosarcoma; a hematopoietic malignancy such as Hodgkin's lymphoma, non-Hodgkin's lymphoma, or leukemia; a neuroectodermal tumor such as peripheral nerve tumor, astrocytoma, or melanoma; or a mesotheliomas.

Another aspect of the invention provides a method of determining presence and/or abundance of CD44v6 in a sample from a subject, the method comprising contacting the sample with any of the subject anti-CD44v6 antibody or antigen-binding fragment thereof.

Another aspect of the invention provides a method of determining presence and/or abundance of CD44v9 in a sample from a subject, the method comprising contacting the sample with any of the subject anti-CD44v9 antibody or antigen-binding fragment thereof.

Another aspect of the invention provides a method of diagnosing and treating a subject having cancer, wherein cells of the cancer expresses CD44v6, the method comprising: (1) using the subject method to determine the presence and/or abundance of CD44v6 in a cancer sample from the subject in order to identify subject expressing CD44v6 in the cancer sample; (2) administering to said subject a therapeutically effective amount of any of the subject anti-CD44v6 antibody or antigen-binding fragment thereof, or the polypeptide thereof, or the immunoconjugate thereof, or the pharmaceutical composition thereof; thereby diagnosing and treating the subject having cancer.

Another aspect of the invention provides q method of diagnosing and treating a subject having cancer, wherein cells of the cancer expresses CD44v9, the method comprising: (1) using the subject method to determine the presence and/or abundance of CD44v9 in a cancer sample from the subject in order to identify subject expressing CD44v9 in the cancer sample; (2) administering to said subject a therapeutically effective amount of any of the subject anti-CD44v9 antibody or antigen-binding fragment thereof, or the polypeptide thereof, or the immunoconjugate thereof, or the pharmaceutical composition thereof; thereby diagnosing and treating the subject having cancer.

It should be understood that any of the embodiments described herein, including those described in Examples only and those described only under one aspect of the invention, can be combined with one or more other embodiments, unless explicitly disclaimed or improper.

### BRIEF DESCRIPTION OF THE DRAWINGS

FIG. 1A is a schematic drawing of live-cell MabArray for isolating the CD44v6 or CD44v9 monoclonal antibody mAb119 or mAb116. Specifically, about 6×10⁴ different monoclonal antibodies (mAbs) were printed onto 4 glass aldehyde chips (75×25 mm) using Arrayjet printer to generate MabArray. The MabArray chips were then blocked with 10% BSA overnight, before the experiments were performed. Live lung cancer cell line PC9 cells were labeled with a green fluorescent nucleic acid stain SYTO14 (ThermoFisher Scientific), and incubated with the chips at a density of 1×10⁷ cells/mL in PBS for 1 hour. MabArray chips were then washed with PBS gently and scanned with Genepix scanner.

FIG. 1B shows images of mAb119 and control mAb in 4 independent PC9 live cell MabArray experiments. Live PC9 cells were captured by mAb119 on MabArray chips.

FIG. 2 shows results of FACs analysis of mAb119 on PC9 cells. PC9 FACS titration of mAb119 was performed by incubating PC9 cells with a serial dilution (30000 pM to 0.1 pM, 3 fold serial dilution) of mAb119 for 30 min on ice, before the cells were stained with Alexa488-conjugated anti-mouse IgG (Jackson lab) for 30 min. MFI was analyzed using BD C6. Affinity K_{D} was determined to be about 2 nM.

FIG. 3 shows that PC9 cells internalized bound mAb119. Live PC cells were cultured on coverslips, and were incubated with 10 μg/mL mAb119 for 1 hr on ice, before the cells were washed 3 times with PBS. Cells were then cultured at 37° C. for 0 hr, 2 hr, or 4 hr, before fixation with 4% PFA before detected with FITC conjugated secondary antibody by FACs. PC9 cells were then co-stained by mAb119 (labeled by a green fluorescent dye Alexa488) and anti-LAMP1 (labeled by a red fluorescent dye Alexa595). Specifically, PC9 cells were permeablized with 0.1% Triton X and incubated with mAb119 and Rabbit anti-LAMP1 antibody (1:200, Abcam) and mAb119 for 1 hr. Antibodies were then labeled with Alexa488 conjugated anti-Mouse antibody and Alexa595 conjugated anti-Rabbit antibody, respectively. Lysosomal-associated membrane protein 1 (LAMP1) is a glycoprotein primarily expressed across lysosomal membranes. Colocalization of mAb119 and anti-LAMP1 signals results in yellow signal that indicates internalization of mAb119 by PC9 cells to the lysosomal compartment. mAb119 was first observed on the cell surface without any co-localization with LAMP1 at 0 hr. Colocalization of mAb119 and LAMP1 was observed at 2 hr and 4 hr.

FACs analysis based on surface fluorescence shows mAb119 internalization on PC9 cells (data not shown). Specifically, live PC9 cells were incubated with 10 μg/mL mAb119 for 0.5 hr on ice before washed 3 times with PBS. Cells were then cultured at 37° C. for 0 hr, 2 hr, or 4 hr, before fixation with 4% PFA. Cells were then stained with Alexa488 conjugated anti-Mouse antibody and analyzed with FACs by calculating surface MFI. Surface MFI, which represented surface localization of mAb119, reduced by 70% and 80% after 2 hr and 4 hr incubation at 37° C., respectively. Shown is the quantification of the FACs data is expressed as the mean percent internalization±SEM (n=3) in PC9 cells. The vertical axis represents the relative surface florescence (%). The data shows that mAb119 could bind membrane antigen and be internalized in PC9 cells.

FIG. 4 shows that the indirect cytotoxicity of mAb119 is antigen expression-dependent. PC9 or TE1 cells were cultured in 96-well plate at 2000 cells/well confluence overnight. Cells were treated with serial dilution of mAb119 together with 2 μg/mL MMAE-conjugated goat anti-mouse IgG antibody for 72 hrs. Cell number was then calculated by CCK8 (dojindo). Different cytotoxicity was observed in TE1 and PC9 cells. The antibody cocktail inhibited PC9 growth with an IC₅₀ of 18 pM, while the same antibody cocktail did not inhibit TE1 cell growth. Shown is the representative data derived from TE1 and PC9 cells, expressed as the mean percent growth inhibition±SEM (n=3).

The expression of mAb119 antigen in the two cell lines were also determined by FACs. The side insert panels show FACs analysis of TE1 (top panel) and PC9 (bottom panel) labeled by mAb119. The results suggest that PC9 cells, but not TE1 cells, express mAb119 antigen. Thus the indirect cytotoxicity was positively correlated with antigen expression.

FIGS. 5A and 5B show that mAb119 targets human CD44 v6 exon. PC9 was transfected with a mixture of 4 different siRNAs targeting human CD44 v6 epitope or control siRNA for 48 hrs. Transfected cells were then either stained with mAb119 and analyzed by FACs, or the total protein was extracted and the abundance of mAb119 antigen was evaluated by Western blotting. Knockdown of CD44v6 decreased mAb119 surface staining intensity in FACs (FIG. 5A, FACs data showing that CD44v6 siRNA (V6.si) inhibits surface signal of mAb119 (representative of n=3)). Knockdown of CD44v6 also decreased protein expression level of the mAb119 antigen (FIG. 5B, Western blotting data showing that the CD44v6 siRNA (V6.si) inhibits protein expression of mAb119 antigen (representative of n=3)). The data suggests that mAb119 targets CD44v6.

FIG. 6A is a schematic drawing of the structure of a mAb119-ADC (AMT119). mAb119 was conjugated with MC-vc-PAB-MMAE.

FIG. 6B is a graph of HPLC-HIC (Hydrophobic Interaction Chromatography) of AMT119. The average drug-antibody ratio (DAR) was about 6.

FIG. 7 shows cytotoxicity of AMT119 in PC9 and TE1 cells. Graphs are representative data derived from PC9 and TE1 cells showing the mean percent growth inhibition±SEM of AMT119 (n=3). The IC₅₀ values were 2,600 pM and 39,000 pM in PC9 and TE1 cells, respectively. The difference was consistent with the different expression levels of CD44v6 in the two cell lines (see FIG. 4).

FIGS. 8A and 8B show expression of CD44v6 in human non-small-cell lung cancer (NSCLC, right panels of FIG. 8A) and normal lung tissues (left panel of FIG. 8A). IHC (immunohistochemistry) detection of CD44v6 protein using mAb119 antibody is shown from a series of normal and cancer tissues, showing that CD44v6 was up-regulated in a tumor-specific manner. Photomicrograph images depict tumor tissues representative of 0, 1+, 2+ and 3+ staining intensity (right panels of FIG. 8A). FIG. 8B shows prevalence of CD44v6 in different subtypes of NSCLC. SCC, Squamous cell carcinoma; LCC, Large cell carcinoma.

FIG. 9 shows results of FACs analysis of mAb116 on PC9 cells. PC9 FACS titration of mAb116 was performed by incubating PC9 cells with a serial dilution (30000 pM to 0.1 pM, 3 fold serial dilution) of mAb116 for 30 min on ice, before the cells were stained with Alexa488-conjugated anti-mouse IgG (Jackson lab) for 30 min. MFI was analyzed using BD C6. Affinity K_{D} was determined to be about 980 pM (or 0.98 nM).

FIG. 10 shows that PC9 cells internalized bound mAb116. Live PC cells were cultured on coverslips, and were incubated with 10 μg/mL mAb116 for 1 hr on ice, before the cells were washed 3 times with PBS. Cells were then cultured at 37° C. for 0 hr, 2 hr, or 4 hr, before fixation with 4% PFA before detected with FITC conjugated secondary antibody by FACs. PC9 cells were then co-stained by mAb116 (labeled by a green fluorescent dye Alexa488) and anti-LAMP1 (labeled by a red fluorescent dye Alexa595). Specifically, PC9 cells were permeablized with 0.1% Triton X and incubated with mAb116 and Rabbit anti-LAMP1 antibody (1:200, Abcam) and mAb116 for 1 hr. Antibodies were then labeled with Alexa488 conjugated anti-Mouse antibody and Alexa595 conjugated anti-Rabbit antibody, respectively. Colocalization of mAb116 and anti-LAMP1 signals results in yellow signal that indicates internalization of mAb116 by PC9 cells to the lysosomal compartment. mAb116 was first observed on the cell surface without any co-localization with LAMP1 at 0 hr. Colocalization of mAb116 and LAMP1 was observed at 2 hr and 4 hr.

FACs analysis based on surface fluorescence shows mAb116 internalization on PC9 cells (data not shown). Specifically, live PC9 cells were incubated with 10 μg/mL mAb116 for 0.5 hr on ice before washed 3 times with PBS. Cells were then cultured at 37° C. for 0 hr, 2 hr, or 4 hr, before fixation with 4% PFA. Cells were then stained with Alexa488 conjugated anti-Mouse antibody and analyzed with FACs by calculating surface MFI. Surface MFI, which represented surface localization of mAb116, reduced by about 90% at 4 hr incubation at 37° C. Shown in quantification of the FACs data, expressed as the mean percent internalization±SEM (n=3) in PC9 cells. The vertical axis represents the relative surface florescence (MFI, %). The data shows that mAb116 could bind membrane antigen and be internalized in PC9 cells.

FIG. 11 shows indirect cytotoxicity of mAb116 and control IgG. PC9 cells were cultured in 96-well plate at 2000 cells/well confluence overnight. Cells were then treated with serial dilution of mAb116 or IgG together with 2 μg/mL MMAE-conjugated goat anti mouse IgG antibody for 72 hrs. Cell number was then calculated by CCK8 (dojindo). The mAb116 antibody cocktail inhibited PC9 growth with an IC₅₀ of about 30 pM, but IgG cocktail did not have any effect. Shown is representative data derived from PC9 cells, expressed as the mean percent growth inhibition±SEM (n=3).

FIGS. 12A and 12B show that mAb116 targets human CD44 v9 exon. PC9 was transfected with siRNA targeting human CD44 V9 epitope or control siRNA for 48 hrs. Transfected cells were then either stained with mAb116 and analyzed by FACs, or the total protein was extracted and the abundance of mAb116 antigen was evaluated by Western blotting. Knockdown of CD44v9 decreased mAb116 surface staining intensity in FACs (FIG. 12A, FACs data showing that CD44v9 siRNA (V9.si) inhibits surface signal of mAb116 (representative of n=3). Knockdown of CD44v9 also decreased protein expression level of the mAb116 antigen (FIG. 12B). The data suggests that mAb116 targets CD44v9.

FIG. 13A is a schematic drawing of the structure of a mAb116-ADC (AMT116). mAb116 was conjugated with MC-vc-PAB-MMAE.

FIG. 13B is a graph of HPLC-HIC of AMT116. The average drug-antibody ratio (DAR) was about 4.23.

FIG. 14 shows cytotoxicity of AMT116 in PC9 and KYSE-150 (Esophagus Carcinoma cell line) cells. Graphs are representative data derived from PC9 and KYSE-150 cells showing the mean percent growth inhibition±SEM of AMT116 and IgG control (n=3). The IC₅₀ values of AMT116 were 134 pM and 670.2 pM in PC9 and KYSE-150 cells, respectively.

FIG. 15 shows in vivo efficacy of AMT116. About 5×10⁶ KYSE-150 cells were suspended in 1:1 Matrigel before injection into the right flank of female Balb/c nude mice (8-10 weeks, 20-22 g). Tumor volume (measured by 0.5×length×width²) and body weight were determined at least twice weekly. Mice were grouped randomly (n=5/group) based on their initial tumor size (median tumor volume of approximately 250-500 mm³) before dosing. Vehicle (PBS), AMT116, or control ADC was administered by i.v. infusion (3 mg/kg, q3d×3). Group mean (±SEM) tumor volumes were plotted over the duration of study.

FIGS. 16A and 16B show expression of CD44v9 in human non-small-cell lung cancer (right panels of FIG. 16A) and normal lung tissues (left panel of FIG. 16A). IHC detection of CD44v9 protein using mAb116 antibody is shown from a series of normal and cancer tissues, showing that CD44v9 was up-regulated in a tumor-specific manner. Photomicrograph images depict tumor tissues representative of 0, 1+, 2+ and 3+ staining intensity (right panels of FIG. 16A). FIG. 16B shows prevalence of CD44v9 in different subtypes of NSCLC. SCC, Squamous cell carcinoma; LCC, Large cell carcinoma.

FIG. 17 shows overexpression of CD44v9 in multiple tumor types. IHC detection of CD44v9 protein using mAb116 antibody is shown from a series of normal and cancer tissues, showing that CD44v9 was up-regulated in a tumor-specific manner.

### DETAILED DESCRIPTION OF THE INVENTION

### 1. Overview

The invention described herein is partly based on the finding that certain anti-CD44v6 or anti-CD44v9 antibodies, such as the ones described herein, are effective to treat diseases such as cancer.

Thus one aspect of the invention provides an isolated monoclonal antibody, or an antigen-binding fragment thereof, specific for an isolated CD44v6 epitope, wherein said CD44v6 epitope: (1) comprises/consists essentially of SEQ ID NO: 19 (e.g., an epitope consisting of SEQ ID NO: 19 plus 1 or 2 residues on the N-terminus of SEQ ID NO: 19, SEQ ID NO: 19 plus 1 or 2 residues on the C-terminus of SEQ ID NO: 19, or SEQ ID NO: 19 plus 1 or 2 residues on both the N-terminus and the C-terminus of SEQ ID NO: 19), or (2) consists of SEQ ID NO: 19.

For example, the antibody or antigen-binding fragment of the invention can be raised against the isolated CD44v6 epitope, or raised against a fusion protein or chemical conjugate thereof comprising said isolated CD44v6 epitope and a carrier protein, according to methods known in the art (see below).

In certain embodiments, the anti-CD44v6 monoclonal antibody comprises: (1) a heavy chain variable region (HCVR), comprising a HCVR CDR1 sequence of SEQ ID NO: 1, a HCVR CDR2 sequence of SEQ ID NO: 2, and/or a HCVR CDR3 sequence of SEQ ID NO: 3; and/or (2) a light chain variable region (LCVR), comprising a LCVR CDR1 sequence of SEQ ID NO: 10, a LCVR CDR2 sequence of SEQ ID NO: 11, and/or a LCVR CDR3 sequence of SEQ ID NO: 12.

In certain embodiments, the CD44v6 epitope is SEQ ID NO: 19.

In certain embodiments, the CD44v6 epitope consists essentially of SEQ ID NO: 19 (e.g., an epitope consisting of SEQ ID NO: 19 plus 1 or 2 residues on the N-terminus of SEQ ID NO: 19, SEQ ID NO: 19 plus 1 or 2 residues on the C-terminus of SEQ ID NO: 19, or SEQ ID NO: 19 plus 1 or 2 residues on both the N-terminus and the C-terminus of SEQ ID NO: 19). The added/extra residues at the N- and/or C-terminus of SEQ ID NO: 19 may be naturally occurring in wild-type CD44v6, or may be artificial.

In certain embodiments, the CD44v6 epitope is SEQ ID NO: 24 (HEGYRQTPKEDS).

In certain embodiments, (i) the HCVR further comprises one or more of SEQ ID NOs: 7-9; and/or, (ii) the LCVR further comprises one or more of SEQ ID NOs: 13-18.

In certain embodiments, the isolated anti-CD44v6 monoclonal antibody or antigen-binding fragment thereof binds to said CD44v6 epitope, or a cell having said CD44v6 epitope, with a K_{D} of about 10 nM, about 5 nM, or about 2 nM or less.

In certain embodiments, the isolated monoclonal antibody or antigen-binding fragment thereof is a human-mouse chimeric antibody, a humanized antibody, a human antibody, a CDR-grafted antibody, or a resurfaced antibody.

In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In a related aspect, the invention provides an isolated monoclonal antibody, or an antigen-binding fragment thereof, wherein said isolated monoclonal antibody or antigen-binding fragment thereof binds to the same epitope of CD44v6 that is bound by a reference monoclonal antibody, or competes with said reference monoclonal antibody for binding to the same epitope of CD44v6, wherein said reference monoclonal antibody comprises: (1) a heavy chain variable region (HCVR), comprising a HCVR CDR1 sequence of SEQ ID NO: 1, a HCVR CDR2 sequence of SEQ ID NO: 2, and/or a HCVR CDR3 sequence of SEQ ID NO: 3; (2) a light chain variable region (LCVR), comprising a LCVR CDR1 sequence of SEQ ID NO: 10, a LCVR CDR2 sequence of SEQ ID NO: 11, and/or a LCVR CDR3 sequence of SEQ ID NO: 12.

Another aspect of the invention provides an isolated monoclonal antibody, or an antigen-binding fragment thereof, specific for an isolated CD44v9 epitope, wherein said CD44v9 epitope: (1) comprises/consists essentially of SEQ ID NO: 43 (e.g., an epitope consisting of SEQ ID NO: 43 plus 1 or 2 residues on the N-terminus of SEQ ID NO: 43, SEQ ID NO: 19 plus 1 or 2 residues on the C-terminus of SEQ ID NO: 43, or SEQ ID NO: 43 plus 1 or 2 residues on both the N-terminus and the C-terminus of SEQ ID NO: 43), or (2) consists of SEQ ID NO: 43.

In certain embodiments, the anti-CD44v9 antibody or antigen-binding fragment thereof is raised against said isolated CD44v9 epitope, or raised against a fusion protein or chemical conjugate thereof comprising said isolated CD44v9 epitope and a carrier protein (such as albumin, preferably BSA or ovalbumin, or keyhole limpet hemocyanin (KLH)).

In certain embodiments, the monoclonal antibody comprises: (1) a heavy chain variable region (HCVR), comprising a HCVR CDR1 sequence of SEQ ID NO: 25, a HCVR CDR2 sequence of SEQ ID NO: 26, and/or a HCVR CDR3 sequence of SEQ ID NO: 27; and/or (2) a light chain variable region (LCVR), comprising a LCVR CDR1 sequence of SEQ ID NO: 34, a LCVR CDR2 sequence of SEQ ID NO: 35, and/or a LCVR CDR3 sequence of SEQ ID NO: 36.

In certain embodiments, the CD44v9 epitope is SEQ ID NO: 43.

In certain embodiments, the CD44v9 epitope consists essentially of SEQ ID NO: 43 (e.g., an epitope consisting of SEQ ID NO: 43 plus 1 or 2 residues on the N-terminus of SEQ ID NO: 43, SEQ ID NO: 43 plus 1 or 2 residues on the C-terminus of SEQ ID NO: 43, or SEQ ID NO: 43 plus 1 or 2 residues on both the N-terminus and the C-terminus of SEQ ID NO: 43). The added/extra residues at the N- and/or C-terminus of SEQ ID NO: 43 may be naturally occurring in wild-type CD44v9, or may be artificial.

In certain embodiments, the CD44v9 epitope is SEQ ID NO: 44 (SHEGLEEDKDH).

In certain embodiments, (i) the HCVR further comprises one or more of SEQ ID NOs: 28-33; and/or, (ii) the LCVR further comprises one or more of SEQ ID NOs: 37-42.

In certain embodiments, the isolated monoclonal antibody or antigen-binding fragment thereof binds to said CD44v9 epitope, or a cell having said CD44v9 epitope, with a K_{D} of about 10 nM, about 5 nM, about 2 nM, about 1 nM or less.

In certain embodiments, the isolated monoclonal antibody or antigen-binding fragment thereof is a human-mouse chimeric antibody, a humanized antibody, a human antibody, a CDR-grafted antibody, or a resurfaced antibody.

In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In a related aspect, the invention provides an isolated monoclonal antibody, or an antigen-binding fragment thereof, wherein said isolated monoclonal antibody or antigen-binding fragment thereof binds to the same epitope of CD44v9 that is bound by a reference monoclonal antibody, or competes with said reference monoclonal antibody for binding to the same epitope of CD44v9, wherein said reference monoclonal antibody comprises: (1) a heavy chain variable region (HCVR), comprising a HCVR CDR1 sequence of SEQ ID NO: 25, a HCVR CDR2 sequence of SEQ ID NO: 26, and/or a HCVR CDR3 sequence of SEQ ID NO: 27; (2) a light chain variable region (LCVR), comprising a LCVR CDR1 sequence of SEQ ID NO: 34, a LCVR CDR2 sequence of SEQ ID NO: 35, and/or a LCVR CDR3 sequence of SEQ ID NO: 36.

Another aspect of the invention provides a polypeptide comprising the HCVR and/or the LCVR of any one of the subject anti-CD44v6 or anti-CD44v9 antibodies or antigen binding fragments thereof.

In certain embodiments, the polypeptide is a fusion protein (such as a chimeric antigen T cell receptor).

Chimeric antigen T cell receptor (CAR-T) is also known as chimeric antigen receptor (CAR), chimeric immunoreceptor, chimeric T cell receptor, or artificial T cell receptor. It is engineered receptor that grafts an arbitrary specificity onto an immune effector T cell. Typically, these receptors are used to graft the specificity of a monoclonal antibody onto a T cell, with transfer of their coding sequence facilitated by retroviral vectors. The receptors are called chimeric because they are composed of parts from different sources. CAR-T may be used in treating cancer using adoptive cell transfer in which T cells are removed from a patient and modified so that they express receptors specific to the patient's particular cancer, such as CD44v6 or CD44v9 expressed on cancer cells. The T cells, which can then recognize and kill the cancer cells, are reintroduced into the patient. Modification of T-cells sourced from donors other than the patient may also be used similarly.

In certain embodiments, the CAR-T of the subject invention is a fusion of a subject single-chain variable fragments (scFv) derived from any of the subject monoclonal anti-CD44v6 or anti-CD44v9 antibody, fused to a transmembrane domain (such as the CD3-zeta transmembrane domain) and an endodomain (such as the CD3-zeta endodomain).

In certain embodiments, the scFv is preceded by a signal peptide to direct the nascent protein to the endoplasmic reticulum and subsequent surface expression. Any eukaryotic signal peptide sequence may be used. In certain embodiments, the signal peptide natively attached to the amino-terminal is used (e.g., in a scFv with orientation light chain-linker-heavy chain, the native signal of the light-chain is used).

In certain embodiments, a flexible spacer is added to allow the scFv to orient in different directions to enable optimal antigen binding. The spacer is preferably flexible enough to allow the antigen binding domain to orient in different directions to facilitate antigen recognition. In certain embodiments, the hinge region from IgG1 is used as the spacer. In certain embodiments, the CH₂CH₃ region of immunoglobulin and portions of CD3 is used as the spacer. For most scFv based constructs, the IgG1 hinge usually suffices.

In certain embodiments, the construct comprises a transmembrane domain that is a typical hydrophobic alpha helix derived from the original molecule of the signalling endodomain that protrudes into the cell and transmits the desired signal. In certain embodiments, the transmembrane domain from the most membrane proximal component of the endodomain, such as the CD3-zeta transmembrane domain, is used.

In certain embodiments, the endodomain is the CD3-zeta endodomain containing 3 ITAMs, which transmits an activation signal to the T cell after the antigen is bound by the antigen binding fragment of the invention.

In certain embodiments, the endodomain further comprises intracellular signaling domains from a costimulatory protein receptor (e.g., that of CD28, 41BB, ICOS) fused to the cytoplasmic tail (N- or C-terminal to the CD3-zeta domain) of the construct to provide additional signals to the T cell.

In certain embodiments, the endodomain combines multiple signaling domains, such as CD3z-CD28-41BB or CD3z-CD28-OX40, to augment potency, or to transmit a proliferative/survival signal.

In certain embodiments, the chimeric antigen receptor of the invention further comprises a Strep-tag II sequence (an eight-residue minimal peptide sequence (Trp-Ser-His-Pro-Gln-Phe-Glu-Lys) that exhibits intrinsic affinity toward streptavidin), to provides engineered T cells with an identification marker for rapid purification.

Another aspect of the invention provides a polynucleotide encoding any of the subject polypeptides.

Another aspect of the invention provides a vector comprising any of the subject polynucleotides.

In certain embodiments, the vector is an expression vector (e.g., a mammalian expression vector, a yeast expression vector, an insect expression vector, or a bacterial expression vector).

Another aspect of the invention provides a cell comprising any of the subject anti-CD44v6 or anti-CD44v9 antibody or antigen-binding fragment thereof, any of the subject polypeptide, any of the subject polynucleotide, or any of the subject vector.

In certain embodiments, the cell expresses any of the subject antibody or antigen-binding fragment thereof, or any of the subject polypeptide.

In certain embodiments, the cell is a BHK cell, a CHO cell, or a COS cell.

In certain embodiments, the cell comprises any of the subject anti-CD44v6 or anti-CD44v9 antibody or antigen-binding fragment thereof, or any of the subject polypeptide, on the surface of the cell.

In certain embodiments, the cell is a T-cell bearing a chimeric antigen receptor (CAR-T cell) comprising any of the subject antibody or antigen-binding fragment thereof, or any of the subject polypeptide.

In certain embodiments, viral vectors such as retrovirus, lentivirus or transposon may be used to integrate the transgene bearing the subject CAR-T construct into the host cell genome.

In certain embodiments, non-integrating vectors or episomal DNA/RNA constructs, such as plasmids or mRNA, can be used instead.

In certain embodiments, a vector that is stably maintained in the T cell without being integrated into the genome is used to enable long-term transgene expression without the risk of insertional mutagenesis or genotoxicity.

Another aspect of the invention provides an isolated CD44v6 epitope comprising/consisting essentially of SEQ ID NO: 19 (e.g., an epitope consisting of SEQ ID NO: 19 plus 1 or 2 residues on the N-terminus of SEQ ID NO: 19, SEQ ID NO: 19 plus 1 or 2 residues on the C-terminus of SEQ ID NO: 19, or SEQ ID NO: 19 plus 1 or 2 residues on both the N-terminus and the C-terminus of SEQ ID NO: 19), or consisting of SEQ ID NO: 19.

Another aspect of the invention provides a fusion protein or chemical conjugate comprising the isolated CD44v6 epitope of claim 28, and a carrier protein (such as albumin, preferably BSA or ovalbumin, or keyhole limpet hemocyanin (KLH)).

Another aspect of the invention provides an isolated CD44v9 epitope comprising/consisting essentially of SEQ ID NO: 43 (e.g., an epitope consisting of SEQ ID NO: 43 plus 1 or 2 residues on the N-terminus of SEQ ID NO: 43, SEQ ID NO: 43 plus 1 or 2 residues on the C-terminus of SEQ ID NO: 43, or SEQ ID NO: 43 plus 1 or 2 residues on both the N-terminus and the C-terminus of SEQ ID NO: 43), or consisting of SEQ ID NO: 43.

Another aspect of the invention provides a fusion protein or chemical conjugate comprising the isolated CD44v9 epitope of claim 28a, and a carrier protein (such as albumin, preferably BSA or ovalbumin, or keyhole limpet hemocyanin (KLH)).

As is known in the art, a carrier protein is any protein used for coupling with peptides or other haptens that are not sufficiently large or complex on their own to induce an immune response and produce antibodies. The carrier protein, because it is large and complex, confers immunogenicity to the conjugated hapten, resulting in antibodies being produced against epitopes on the hapten and carrier.

Many proteins can be used as carriers and are chosen based on immunogenicity, solubility, and availability of useful functional groups through which conjugation with the hapten can be achieved. In certain embodiments, the carrier protein used in the instant invention is keyhole limpet hemocyanin (KLH) or an albumin, such as bovine serum albumin (BSA) or ovalbumin.

Many such carrier proteins that can be used in the instant invention are commercially available, such as the Thermo Scientific Imject Mariculture Keyhole Limpet Hemocyanin (mcKLH); the Blue Carrier® Protein (a purified preparation of Concholepas concholepas hemocyanin (CCH) which exhibits most of the same immunogenic properties as the KLH); the Thermo Scientific Imject BSA (a highly purified (i.e., Fraction V) bovine serum albumin); cationized bovine serum albumin (cBSA) (prepared by modifying native BSA with excess ethylenediamine, essentially capping all negatively-charged carboxyl groups with positively-charged primary amines, resulting in a highly positively-charged protein (pI>11) that has significantly increased immunogenicity compared to native BSA); and ovalbumin.

The CD44v6 and CD44v9 epitopes of the invention can be fused to the carrier protein, or chemically conjugated to the carrier protein through, for example, any one or more of the surface primary amine groups of the carrier protein.

Different approaches are available for conjugating haptens/peptide epitopes to carrier proteins, depending on the functional groups available on the hapten/epitope, the required hapten/epitope orientation and distance from the carrier, and the possible effect of conjugation on biological and antigenic properties. For example, epitopes having primary amines (the N-terminus and the side chain of lysine residues), carboxylic groups (C-terminus or the side chain of aspartic acid and glutamic acid), and sulfhydryls (side chain of cysteine residues) can be targeted for conjugation using such groups. Generally, it is the many primary amines in a carrier protein that are used to couple haptens via a crosslinking reagent.

In certain embodiments, the protein-carrier and peptide-carrier conjugation is carried out using the carbodiimide crosslinker EDC (i.e., EDC conjugation via carboxyl and amine crosslinking).

In certain embodiments, the protein-carrier and peptide-carrier conjugation is carried out using maleimide conjugation (sulfhydryl crosslinking).

In certain embodiments, the protein-carrier and peptide-carrier conjugation is carried out using glutaraldehyde conjugation (amine-to-amine crosslinking).

Another aspect of the invention provides a method of producing any of the subject anti-CD44v6 or anti-CD44v9 antibody or antigen-binding fragment thereof, or any of the subject polypeptide, comprising: (a) culturing any of the subject cell; and, (b) isolating said antibody, antigen-binding fragment thereof, or polypeptide from said cultured cell.

In certain embodiments, the cell is a eukaryotic cell.

Another aspect of the invention provides an immunoconjugate (or antibody-drug conjugate or ADC) having the following formula: Ab-[-L-D]ₙ, wherein: Ab is any of the subject anti-CD44v6 or anti-CD44v9 antibody or antigen-binding fragment thereof, or any of the subject polypeptide thereof, that is covalently linked to one or more units of linker-drug moieties -[-L-D], wherein L is a linker and D is a cytotoxic drug; and, n is an integer from 1 to 20 (e.g., from 1-12); and wherein each linker-drug moiety may have the same or different linker L or cytotoxic drug D.

In certain embodiments, each linker-drug moiety -[-L-D] is covalently linked to Ab via a sidechain amino group of Lys.

In certain embodiments, each linker-drug moiety -[-L-D] is covalently linked to Ab via a sidechain thiol group of Cys.

In certain embodiments, each linker-drug moiety -[-L-D] is covalently linked to Ab via a site-specifically incorporated non-natural amino acid.

In certain embodiments, each linker L comprises a peptide unit.

In certain embodiments, the peptide unit comprises 2, 3, 4, 5, 6, 7, 8, 9, 10, 2-10, or 2-5 amino acid residues.

In certain embodiments, the linker L is non-cleavable by protease (e.g., cathepsin).

In certain embodiments, the linker L is a cleavable linker cleavable by protease (e.g., cathepsin), acidic environment, or redox state change.

In certain embodiments, the cytotoxic drug is a DNA intercalating agent, a microtubule binder, a topoisomerase I inhibitor, or a DNA minor groove binder.

In certain embodiments, the cytotoxic drug is auristatin class such as monomethyl auristatin E (MMAE) and MMAF, maytansine class such as DM-1, DM-3, DM-4, calicheamicin such as ozogamicin, SN-38, or PBD (pyrrolobenzodiazepin).

In a related aspect, the D moiety is not a drug molecule per se, but an adaptor molecule (such as FITC) that can be tightly bound by a universal CAR-T specific for the adaptor molecule. According to this aspect of the invention, a single universal CAR-T cell, which binds with extraordinarily high affinity to an adaptor molecule such as FITC, are used to treat various cancer types when co-administered with bispecific SMDC (small molecule drug conjugate) adaptor molecules. These unique bispecific adaptors are constructed with an adaptor, such as FITC molecule, and a tumor-homing molecule, such as the antigen-binding fragment of the subject anti-CD44v6 or anti-CD44v9 antibody, to precisely bridge the universal CAR-T cell with the cancer cells, which causes localized T cell activation. Anti-tumor activity is induced only when both the universal CAR-T cells and the correct antigen-specific adaptor molecules are present. Anti-tumor activity and toxicity can be controlled further by adjusting the administered adaptor molecule dosing. Treatment of antigenically heterogeneous tumors can be achieved by administration of a mixture of the desired antigen-specific adaptors.

Another aspect of the invention provides a pharmaceutical composition comprising any of the subject anti-CD44v6 or anti-CD44v9 antibody or antigen-binding fragment thereof, or the polypeptide thereof, or the immunoconjugate thereof, and a pharmaceutically acceptable carrier or excipient.

Another aspect of the invention provides a method for inhibiting the growth of a cell expressing CD44v6, comprising contacting the cell with any of the subject anti-CD44v6 antibody or antigen-binding fragment thereof, or the subject polypeptide thereof, or the subject immunoconjugate thereof, or the subject pharmaceutical composition thereof.

In certain embodiments, the cell is a tumor cell.

In certain embodiments, the tumor cell is from a lung cancer (such as NSCLC).

In certain embodiments, the tumor cell is from colorectal cancer, breast cancer, head and neck cancer, ovarian cancer, bladder cancer, pancreatic cancer, or metastatic cancers of the brain.

Another aspect of the invention provides a method for inhibiting the growth of a cell expressing CD44v9, comprising contacting the cell with any of the subject anti-CD44v9 antibody or antigen-binding fragment thereof, or the polypeptide thereof, or the immunoconjugate thereof, or the pharmaceutical composition thereof.

In certain embodiments, the cell is a tumor cell.

In certain embodiments, the tumor cell is from a lung cancer (such as NSCLC).

In certain embodiments, the tumor cell is from colorectal cancer, breast cancer, liver, head and neck cancer, ovarian cancer, bladder cancer, pancreatic cancer, or metastatic cancers of the brain.

Another aspect of the invention provides a method for treating a subject having cancer, wherein cells of the cancer expresses CD44v6, the method comprising administering to said subject a therapeutically effective amount of an antagonist of CD44v6 comprising a CD44v6 antibody or an antigen-binding fragment thereof.

Another aspect of the invention provides a method for treating a cell-proliferative disorder in a subject, wherein cells of the cell-proliferative disorder expresses CD44v6, the method comprising administering to said subject a therapeutically effective amount of an antagonist of CD44v6 comprising a CD44v6 antibody or an antigen-binding fragment thereof.

In certain embodiments, the antagonist of CD44v6 comprises any of the subject anti-CD44v6 antibody or antigen-binding fragment thereof, or the subject polypeptide thereof, or the subject immunoconjugate thereof, or the subject pharmaceutical composition thereof.

In certain embodiments, the cancer is an epithelial carcinoma including breast, lung, liver, colorectal, head and neck, esophageal, pancreatic, ovarian, bladder, gastric, skin, endometrial, ovarian, testicular, esophageal, prostatic or renal origin; a bone and soft-tissue sarcoma such as osteosarcoma, chondrosarcoma, fibrosarcoma, malignant fibrous histiocytoma (MFH), leiomyosarcoma; a hematopoietic malignancy such as Hodgkin's lymphoma, non-Hodgkin's lymphoma, or leukemia; a neuroectodermal tumor such as peripheral nerve tumor, astrocytoma, or melanoma; or a mesotheliomas.

Another aspect of the invention provides a method for treating a subject having cancer, wherein cells of the cancer expresses CD44v9, the method comprising administering to said subject a therapeutically effective amount of an antagonist of CD44v9 comprising a CD44v9 antibody or an antigen-binding fragment thereof.

Another aspect of the invention provides a method for treating a cell-proliferative disorder in a subject, wherein cells of the cell-proliferative disorder expresses CD44v9, the method comprising administering to said subject a therapeutically effective amount of an antagonist of CD44v9 comprising a CD44v9 antibody or an antigen-binding fragment thereof.

In certain embodiments, the antagonist of CD44v9 comprises any of the subject anti-CD44v9 antibody or antigen-binding fragment thereof, or the polypeptide thereof, or the immunoconjugate thereof, or the pharmaceutical composition thereof.

In certain embodiments, the cancer is an epithelial carcinoma including breast, lung, liver, colorectal, head and neck, esophageal, pancreatic, ovarian, bladder, gastric, skin, endometrial, ovarian, testicular, esophageal, prostatic or renal origin; a bone and soft-tissue sarcoma such as osteosarcoma, chondrosarcoma, fibrosarcoma, malignant fibrous histiocytoma (MFH), leiomyosarcoma; a hematopoietic malignancy such as Hodgkin's lymphoma, non-Hodgkin's lymphoma, or leukemia; a neuroectodermal tumor such as peripheral nerve tumor, astrocytoma, or melanoma; or a mesotheliomas.

Another aspect of the invention provides a method of determining presence and/or abundance of CD44v6 in a sample from a subject, the method comprising contacting the sample with any of the subject anti-CD44v6 antibody or antigen-binding fragment thereof.

Another aspect of the invention provides a method of determining presence and/or abundance of CD44v9 in a sample from a subject, the method comprising contacting the sample with any of the subject anti-CD44v9 antibody or antigen-binding fragment thereof.

Another aspect of the invention provides a method of diagnosing and treating a subject having cancer, wherein cells of the cancer expresses CD44v6, the method comprising: (1) using the subject method to determine the presence and/or abundance of CD44v6 in a cancer sample from the subject in order to identify subject expressing CD44v6 in the cancer sample; (2) administering to said subject a therapeutically effective amount of any of the subject anti-CD44v6 antibody or antigen-binding fragment thereof, or the polypeptide thereof, or the immunoconjugate thereof, or the pharmaceutical composition thereof; thereby diagnosing and treating the subject having cancer.

Another aspect of the invention provides q method of diagnosing and treating a subject having cancer, wherein cells of the cancer expresses CD44v9, the method comprising: (1) using the subject method to determine the presence and/or abundance of CD44v9 in a cancer sample from the subject in order to identify subject expressing CD44v9 in the cancer sample; (2) administering to said subject a therapeutically effective amount of any of the subject anti-CD44v9 antibody or antigen-binding fragment thereof, or the polypeptide thereof, or the immunoconjugate thereof, or the pharmaceutical composition thereof; thereby diagnosing and treating the subject having cancer.

With the invention generally described above, certain specific aspects or embodiments of the invention are described further in the sections below.

### 2. Definitions

The terms “antibody,” “antibody molecule,” and “antibody protein” are used interchangeably herein and shall be considered equivalent. They include an immunoglobulin molecule that recognizes and specifically binds to a target molecule, such as a protein, polypeptide, peptide, carbohydrate, polynucleotide, lipid, or combinations of the foregoing through at least one antigen recognition site within the light chain and/or heavy chain variable regions of the immunoglobulin molecule. As used herein, the term “antibody” encompasses intact polyclonal antibodies, intact monoclonal antibodies, and may as an abbreviation include antibody fragments (such as Fab, Fab′, F(ab′)₂, and Fv fragments), single chain Fv (scFv) mutants, multispecific antibodies such as bispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

In some embodiments, an antibody is a non-naturally occurring, recombinantly generated antibody. In some embodiments, an antibody is purified from natural components. In some embodiments, an antibody is recombinantly produced. In some embodiments, an antibody is produced by a hybridoma, or generated in a library of antibodies.

“Complementarity determining regions (CDRs) of a monoclonal antibody” are understood to be those amino acid sequences involved in specific antigen binding according to Kabat (Kabat E. A., Wu T. T., Perry H. M., Gottesman K. S. and Foeller C. (1991) Sequences of Proteins of Immunological Interest (5th Ed.). NIH Publication No. 91-3242. U.S. Department of Health and Human Services, Public Health Service, National Institutes of Health, Bethesda, Md., incorporated herein by reference) in connection with Chothia and Lesk (Chothia and Lesk (1987) J. Mol. Biol. 196:901-917, incorporated herein by reference).

As used herein, the term “framework modifications” refers to the exchange, deletion or addition of single or multiple amino acids in the variable regions surrounding the individual complementarity determining regions. Framework modifications may have an impact on the immunogenicity, producibility or binding specificity of an antibody protein.

An “antigen-binding fragment,” “antigen-binding portion,” or “fragment” for short, as used herein, refers to a shorter version of the antibody molecule, i.e. any polypeptide subset, characterized in that it is encoded by a shorter nucleic acid molecule than the full length sequence, but still retains its antibody binding activity (e.g., substantially the same binding specificity, although can be slightly worse binding affinity as measured by K_{d}).

These terms refer to a portion of an intact antibody and refer to the antigenic determining variable regions of an intact antibody. Examples of antibody fragments include, but are not limited to, Fab, Fab′, F(ab′)₂, and Fᵥ fragments, linear antibodies, single chain antibodies, and multispecific antibodies formed from antibody fragments. The term “antigen-binding fragment” of an antibody includes one or more fragments of an antibody that retain the ability to specifically bind to an antigen. It has been shown that the antigen-binding function of an antibody can be performed by certain fragments of a full-length antibody. Examples of binding fragments encompassed within the term “antigen-binding fragment” of an antibody include (without limitation): (i) an Fab fragment, a monovalent fragment consisting of the V_{L}, V_{H}, C_{L}, and C_{H}1 domains (e.g., an antibody digested by papain yields three fragments: two antigen-binding Fab fragments, and one Fc fragment that does not bind antigen); (ii) a F(ab′)₂ fragment, a bivalent fragment comprising two Fab fragments linked by a disulfide bridge at the hinge region (e.g., an antibody digested by pepsin yields two fragments: a bivalent antigen-binding F(ab′)₂ fragment, and a pFc′ fragment that does not bind antigen) and its related F(ab′) monovalent unit; (iii) a F_{d} fragment consisting of the V_{H} and C_{H}1 domains (i.e., that portion of the heavy chain which is included in the Fab); (iv) a Fᵥ fragment consisting of the V_{L} and V_{H} domains of a single arm of an antibody, and the related disulfide linked Fᵥ; (v) a dAb (domain antibody) or sdAb (single domain antibody) fragment (Ward et al., Nature 341:544-546, 1989), which consists of a V_{H} domain; and (vi) an isolated complementarity determining region (CDR).

Various techniques are known for the production of antibody fragments. Traditionally, these fragments are derived via proteolytic digestion of intact antibodies (for example Morimoto et al., Journal of Biochemical and Biophysical Methods 24: 107-117, 1993; Brennan et al., Science 229:81, 1985). In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from antibody phage libraries. The antibody fragment can also be linear antibodies as described in U.S. Pat. No. 5,641,870, for example, and can be monospecific or bispecific. Other techniques for the production of antibody fragments will be apparent to the skilled practitioner.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)₂, Fᵥ), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

Monoclonal antibodies can be prepared using hybridoma methods, such as those described by Kohler and Milstein (1975) Nature 256:495. Using the hybridoma method, a mouse, hamster, or other appropriate host animal, is immunized to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies.

Alternatively monoclonal antibodies can also be made using recombinant DNA methods as described in U.S. Pat. No. 4,816,567. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., Nature 348:552-554, 1990; Clackson et al., Nature, 352:624-628, 1991; and Marks et al., J. Mol. Biol. 222:581-597, 1991).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody, or, 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

The term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (Jones et al., Nature 321:522-525, 1986; Riechmann et al., Nature 332:323-327, 1988; Verhoeyen et al., Science 239:1534-1536, 1988).

Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing CD44v6 or CD44v9 binding. Accordingly, part or all of the non-human or human CDR sequences are maintained while the non-human sequences of the variable and constant regions can be replaced with human or other amino acids.

Antibodies can also optionally be humanized, resurfaced, engineered or human antibodies engineered with retention of high affinity for the antigen CD44v6 or CD44v9 and other favorable biological properties. To achieve this goal, humanized (or human) or engineered anti-CD44v6 or anti-CD44v9 antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as CD44v6 or CD44v9. In this way, framework (FR) residues can be selected and combined from the consensus and import sequences so that the desired antibody characteristic, such as increased affinity for the target antigen(s), is achieved.

Humanization, resurfacing or engineering of antibodies of the present invention can be performed using any known method, such as but not limited to those described in, Winter (Jones et al., Nature 321:522, 1986; Riechmann et al., Nature 332:323, 1988; Verhoeyen et al., Science 239:1534, 1988, Sims et al., J. Immunol. 151:2296, 1993; Chothia and Lesk, J. Mol. Biol. 196:901, 1987, Carter et al., Proc. Natl. Acad. Sci. U.S.A. 89:4285, 1992; Presta et al., J. Immunol. 151:2623, 1993; Raguska et al., Proc. Natl. Acad. Sci. U.S.A. 91(3):969-973, 1994; U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; PCT/: US98/16280; US96/18978; US91/09630; US91/05939; US94/01234; GB89/01334; GB91/01134; GB92/01755; WO90/14443; WO90/14424; WO90/14430; EP 229246; 7,557,189; 7,538,195; and 7,342,110, each of which is entirely incorporated herein by reference, including the references cited therein.

In certain alternative embodiments, the antibody to CD44v6 or CD44v9 is a human antibody. Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., 1991, J. Immunol, 147 (1):86-95; and U.S. Pat. No. 5,750,373). Also, the human antibody can be selected from a phage library, where that phage library expresses human antibodies, as described, for example, in Vaughan et al., Nat. Biotech. 14:309-314, 1996, Sheets et al., Proc. Nat'l. Acad. Sci. 95:6157-6162, 1998, Hoogenboom and Winter, J. Mol. Biol. 227:381, 1991, and Marks et al., J. Mol. Biol. 222:581, 1991). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., J. Mol. Bio. doi: 10.1016/j.jmb.2007.12.018, 2007 (each of which is incorporated by reference in its entirety). Affinity maturation strategies and chain shuffling strategies (Marks et al., Bio/Technology 10:779-783, 1992, incorporated by reference in its entirety) are known in the art and can be employed to generate high affinity human antibodies.

Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In some instances, the Fᵥ framework region (FR) residues of a human immunoglobulin are replaced with the corresponding residues in an antibody from a non-human species that has the desired specificity, affinity, and capability. The humanized antibody can be further modified by the substitution of additional residues either in the Fᵥ framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (F_{c}), typically that of a human immunoglobulin. Examples of methods used to generate humanized antibodies are described in U.S. Pat. Nos. 5,225,539 and 5,639,641, Roguska et al., Proc. Natl. Acad. Sci. USA 91(3):969-973, 1994; and Roguska et al., Protein Eng. 9(10):895-904, 1996 (all incorporated herein by reference). In some embodiments, a “humanized antibody” is a resurfaced antibody. In some embodiments, a “humanized antibody” is a CDR-grafted antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, 5th ed., 1991, National Institutes of Health, Bethesda Md.); and (2) an approach based on crystallographic studies of antigen-antibody complexes (Al-lazikani et al., J. Molec. Biol. 273:927-948, 1997). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed., Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed., Public Health Service, National Institutes of Health, Bethesda, Md. (1991) (incorporated herein by reference). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917,1987). The end of the Chothia CDR-H1 loop when numbered using the Kabat numbering convention varies between H32 and H34 depending on the length of the loop. This is because the Kabat numbering scheme places the insertions at H35A and H35B—if neither 35A nor 35B is present, the loop ends at 32; if only 35A is present, the loop ends at 33; if both 35A and 35B are present, the loop ends at 34. The AbM hypervariable regions represent a compromise between the Kabat CDRs and Chothia structural loops, and are used by Oxford Molecular's AbM antibody modeling software.

| Loop | Kabat | AbM | Chiothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H9S-H102 | H95-H102 | H95-H102 |

The term “human antibody” means an antibody produced by a human or an antibody having an amino acid sequence corresponding to an antibody produced by a human made using any technique known in the art. In certain embodiments, the human antibody does not have non-human sequence. This definition of a human antibody includes intact or full-length antibodies, or antigen-binding fragments thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid or reduce the chance of eliciting an immune response in that species (e.g., human). In certain embodiments, chimeric antibody may include an antibody or antigen-binding fragment thereof comprising at least one human heavy and/or light chain polypeptide, such as, for example, an antibody comprising murine light chain and human heavy chain polypeptides.

For the purposes of the present invention, it should be appreciated that modified antibodies can comprise any type of variable region that provides for the association of the antibody with the polypeptides of a human CD44v6 or CD44v9. In this regard, the variable region can comprise or be derived from any type of mammal that can be induced to mount a humoral response and generate immunoglobulins against the desired tumor associated antigen. As such, the variable region of the modified antibodies can be, for example, of human, murine, non-human primate (e.g., cynomolgus monkeys, macaques, etc.) or lupine origin. In some embodiments both the variable and constant regions of the modified immunoglobulins are human. In other embodiments the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It may not be necessary to replace all of the CDRs with the complete CDRs from the donor variable region to transfer the antigen-binding capacity of one variable domain to another. Rather, it may only be necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. That is, the modified antibodies disclosed herein can comprise alterations or modifications to one or more of the three heavy chain constant domains (CH1, CH2, or CH3) and/or to the light chain constant domain (CL). In some embodiments, modified constant regions wherein one or more domains are partially or entirely deleted are contemplated. In some embodiments, the modified antibodies will comprise domain deleted constructs or variants wherein the entire CH2 domain has been removed (ACH2 constructs). In some embodiments, the omitted constant region domain will be replaced by a short amino acid spacer (e.g., 10 residues) that provides some of the molecular flexibility typically imparted by the absent constant region.

It will be noted that in certain embodiments, the modified antibodies can be engineered to fuse the CH3 domain directly to the hinge region of the respective modified antibodies. In other constructs it may be desirable to provide a peptide spacer between the hinge region and the modified CH2 and/or CH3 domains. For example, compatible constructs could be expressed wherein the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic, or even omitted altogether, so as to maintain the desired biochemical qualities of the modified antibodies.

Besides the deletion of whole constant region domains, it will be appreciated that the antibodies of the present invention can be provided by the partial deletion or substitution of a few or even a single amino acid. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g., complement C1Q binding) to be modulated. Such partial deletions of the constant regions can improve selected characteristics of the antibody (serum half-life) while leaving other desirable functions associated with the subject constant region domain intact. Moreover, as alluded to above, the constant regions of the disclosed antibodies can be modified, e.g., through the mutation or substitution of one or more amino acids, which may enhance the profile of the resulting construct. In this respect it may be possible to disrupt the activity provided by a conserved binding site (e.g., Fc binding) while substantially maintaining the configuration and immunogenic profile of the modified antibody. Certain embodiments can comprise the addition of one or more amino acids to the constant region to enhance desirable characteristics such as decreasing or increasing effector function or provide for more cytotoxin or carbohydrate attachment. In such embodiments it can be desirable to insert or replicate specific sequences derived from selected constant region domains.

The present invention further embraces variants and equivalents which are substantially homologous to the chimeric, humanized and human antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art, such as those defined hereinabove.

The terms “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}) or the half-maximal effective concentration (EC₅₀). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described herein.

The phrase “substantially similar,” or “substantially the same,” as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristics measured by said values (e.g., K_{d} values). The difference between said two values is less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some embodiments, an antibody, polynucleotide, vector, cell, or composition which is isolated is substantially pure.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

As used herein, “substantially pure” refers to material which is at least 50% pure (i.e., free from contaminants), at least 90% pure, at least 95% pure, at least 98% pure, or at least 99% pure.

“A functional variant” of the antibody molecule according to the invention is an antibody molecule which possesses a biological activity (either functional or structural) that is substantially similar to the antibody molecule according to the invention, i.e. a substantially similar substrate specificity or cleavage of the substrate.

The term “functional variant” also includes “a fragment”, “an allelic variant” “a functional variant”, “variant based on the degenerative nucleic acid code” or “chemical derivatives.” Such a “functional variant” may carry one or several point mutations, one or several nucleic acid exchanges in the coding sequence, deletions or insertions or one or several amino acid exchanges, deletions or insertions. Said functional variant is still retaining its biological activity such as antibody binding activity, at least in part or even going along with an improvement said biological activity.

A “functional variant” of the antibody molecule according to the invention may also include an antibody molecule which possesses a biological activity (either functional or structural) that is substantially similar to the antibody molecule according to the invention, i.e. a substantially similar target molecule binding activity.

An “allelic variant” is a variant due to the allelic variation, e.g. differences in the two alleles in humans. Said variant is still retaining its biological activity such as antibody target binding activity, at least in part or even going along with an improvement said biological activity.

A “variant based on the degenerative of the genetic code” is a variant due to the fact that a certain amino acid may be encoded by several different nucleotide triplets. Said variant is still retaining its biological activity such as antibody binding activity, at least in part or even going along with an improvement said biological activity.

A “fusion molecule” may be the antibody molecule according to the invention fused to e.g. a reporter such as a radiolabel, a chemical molecule such as a toxin or a fluorescent label or any other molecule known in the art.

As used herein, a “chemical derivative” according to the invention is an antibody molecule according to the invention chemically modified or containing additional chemical moieties not normally being part of the molecule. Such moieties may improve the molecule's activity such as target destruction (e.g. killing of tumor cells) or may improve its solubility, absorption, biological half-life etc.

A molecule is “substantially similar” to another molecule if both molecules have substantially similar structures or biological activity. Thus, provided that two molecules possess a similar activity, they are considered variants as that term is used herein even if the structure of one of the molecules is not found in the other, or if the sequence of amino acid residues is not identical.

A “sample” or “biological sample” of the present invention is of biological origin, in specific embodiments, such as from eukaryotic organisms. In some embodiments, the sample is a human sample, but animal samples may also be used. Non-limiting sources of a sample for use in the present invention include solid tissue, biopsy aspirates, ascites, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example. A “cancerous/tumor sample” is a sample that contains a cancerous cell. The method can be used to examine an aspect of expression of CD44v6 or CD44v9 or a state of a sample, including, but not limited to, comparing different types of cells or tissues, comparing different developmental stages, and detecting or determining the presence and/or type of disease or abnormality.

For many uses of the antibodies according to the invention it is desirable to have the smallest possible antigen-binding, i.e., CD44v6- or CD44v9-binding units. Therefore in another preferred embodiment an antibody protein according to the invention is a Fab fragment (Fragment antigen-binding=Fab). These CD44v6-specific antibody proteins according to the invention consist of the variable regions of both chains which are held together by the adjacent constant region. These may be formed by protease digestion, e.g. with papain, from conventional antibodies, but similar Fab fragments may also be produced in the mean time by genetic engineering. In another preferred embodiment an antibody protein according to the invention is an F(ab′)2 fragment, which may be prepared by proteolytic cleaving with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). In another preferred embodiment a CD44v6- or CD44v9-specific antibody molecule according to the invention is such an Fv fragment. Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins of this kind known from the prior art are described in Huston et al. (1988, PNAS 16: 5879-5883). Therefore, in another preferred embodiment an CD44v6- or CD44v9-specific antibody protein according to the invention is a single-chain-Fv protein (scFv).

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerization of the scFv, scFv were prepared as fusion proteins with multimerization domains. The multimerization domains may be, e.g. the CH3 region of an IgG or coiled coil structure (helix structures) such as Leucin-zipper domains. However, there are also strategies in which the interaction between the VH/VL regions of the scFv are used for the multimerisation (e.g. di-, tri- and pentabodies). Therefore in another embodiment an antibody protein according to the invention is an CD44v6- or CD44v9-specific diabody antibody fragment. By diabody the skilled person means a bivalent homodimeric scFv derivative (Hu et al., 1996, PNAS 16: 5879-5883). The shortening of the Linker in an scFv molecule to 5-10 amino acids leads to the formation of homodimers in which an inter-chain VH/NL-superimposition takes place. Diabodies may additionally be stabilised by the incorporation of disulphide bridges. Examples of diabody-antibody proteins from the prior art can be found in Perisic et al. (1994, Structure 2: 1217-1226).

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a Linker region. The disulphide bridges in the Hinge region are mostly formed in higher cells and not in prokaryotes. In another preferred embodiment an antibody protein according to the invention is an CD44v6-specific minibody antibody fragment. Examples of minibody-antibody proteins from the prior art can be found in Hu et al. (1996, Cancer Res. 56: 3055-61).

By triabody the skilled person means a: trivalent homotrimeric scFv nderivative (Kortt et al. 1997 Protein Engineering 10: 423-433). ScFv derivatives wherein VH-VL are fused directly without a linker sequence lead to the formation of trimers.

The skilled person will also be familiar with so-called miniantibodies which have a bi-, tri- or tetravalent structure and are derived from scFv. The multimerization is carried out by di-, tri- or tetrameric coiled coil structures (Pack et al., 1993 Biotechnology II:, 1271-1277; Lovejoy et al. 1993 Science 259: 1288-1293; Pack et al., 1995 J. Mol. Biol. 246: 28-34).

Therefore in one embodiment an antibody protein according to the invention is a CD44v6- or CD44v9-specific multimerized molecule based on the abovementioned antibody fragments and may be, for example, a triabody, a tetravalent miniantibody or a pentabody.

Humanized CD44v6- or CD44v9-specific antibody proteins can be generated by molecular biology methods known in the art.

The variable regions of the antibody proteins of the present invention are typically linked to at least a portion of the immunoglobulin constant region (Fc), typically that of a human immunoglobulin. Human constant region DNA sequences can be isolated in accordance with well-known procedures from a variety of human cells, but preferably immortalized B cells (see Kabat et al., supra, and WO 87/02671). Hence the antibody proteins of the invention may contain all or only a portion of the constant region as long as they exhibit specific binding to the CD44v6 or CD44v9 antigen. The choice of the type and extent of the constant region depends on whether effector functions like complement fixation or antibody dependent cellular toxicity are desired, and on the desired pharmacological properties of the antibody protein. The antibody protein of the invention will typically be a tetramer consisting of two light chain/heavy chain pairs, but may also be dimeric, i.e. consisting of a light chain/heavy chain pair, e.g. a Fab or Fv fragment.

Therefore, in a further embodiment the invention relates to antibody proteins according to the invention, characterized in that they have a variable light chain region and a variable heavy chain region, each joined to a human constant region. In particular, the variable region of the light chain was joined to a human kappa constant region and the variable region of the heavy chain was joined to a human gamma-1 constant region. Other human constant regions for chimerizing light and heavy chains are also available.

Humanization of the variable region of a murine antibody may be achieved employing methods known in the art. EP 0239400 discloses grafting of the CDRs of a murine variable region into the framework of a human variable region. WO 90/07861 discloses methods of reshaping a CDR-grafted variable region by introducing additional framework modifications. WO 92/11018 discloses methods of producing humanized Ig combining donor CDRs with an acceptor framework that has a high homology to the donor framework. WO 92/05274 discloses the preparation of framework mutated antibodies starting from a murine antibody. Further prior art references related to humanization of murine monoclonal antibodies are EP 0368684; EP 0438310; WO 92/07075, or WO 92/22653. All are incorporated herein by reference.

In another embodiment, the invention relates to an antibody molecule according to the invention characterized that each of said variable region of the light chain and said variable region of the heavy chain region is separately joined to a human constant region.

In another embodiment, the invention relates to an antibody molecule according to the invention, wherein said human constant region of the light chain is a human kappa constant region.

In another embodiment, the invention relates to an antibody protein according to the invention, wherein said human constant region of the heavy chain is a human IgG1 constant region.

The antibody proteins of the invention provide a highly specific tool for targeting therapeutic agents to the CD44v6 or CD44v9 antigen. Therefore, in a further aspect, the invention relates to antibody proteins according to the invention, wherein said antibody protein is conjugated to a therapeutic agent, optionally via a linker, in an antibody-drug-conjugate (ADC). Of the many therapeutic agents known in the art, therapeutic agents selected from the group consisting of radioisotopes, toxins, toxoids, inflammatogenic agents, enzymes, antisense molecules, peptides, cytokines, and chemotherapeutic agents are preferred. Among the radioisotopes, gamma, beta and alpha-emitting radioisotopes may be used as a therapeutic agent. β-emitting radioisotopes are preferred as therapeutic radioisotopes. ¹⁸⁶Rhenium, ¹⁸⁸Rhenium, ¹³¹Iodine and ⁹⁰Yttrium have been proven to be particularly useful β-emitting isotopes to achieve localized irradiation and destruction of malignant tumor cells. Therefore, radioisotopes selected from the group consisting of ¹⁸⁶Rhenium, ¹⁸⁸Rhenium, ¹³¹Iodine, and ⁹⁰Yttrium are particularly preferred as therapeutic agents conjugated to the antibody proteins of the invention. For example, for the radioiodination of an antibody of the invention, a method as disclosed in WO 93/05804 may be employed.

The term “immunoconjugate,” “conjugate,” or “ADC” as used herein refers to a compound or a derivative thereof that is linked to a cell binding agent (i.e., an anti-CD44v6 or -CD44v9 antibody or fragment thereof) and is defined by a generic formula: A-L-C, wherein C=cytotoxin, L=linker, and A=cell binding agent (CBA), such as anti-CD44v6 or anti-CD44v9 antibody or antibody fragment. Immunoconjugates can also be defined by the generic formula in reverse order: C-L-A.

A “linker” is any chemical moiety that is capable of linking a compound, usually a drug, such as a cytotoxic agent described herein, to a cell-binding agent such as an anti-CD44v6 or -CD44v9 antibody or a fragment thereof in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Administration “in combination with” one or more further therapeutic agents includes simultaneous (concurrent) and consecutive administration in any order.

The term “pharmaceutical formulation” refers to a preparation which is in such form as to permit the biological activity of the active ingredient to be effective, and which contains no additional components which are unacceptably toxic to a subject to which the formulation would be administered. Such formulation can be sterile.

An “effective amount” of an antibody or immunoconjugate as disclosed herein is an amount sufficient to carry out a specifically stated purpose. An “effective amount” can be determined empirically and in a routine manner, in relation to the stated purpose.

The term “therapeutically effective amount” refers to an amount of an antibody or other drug effective to “treat” a disease or disorder in a subject or mammal. In the case of cancer, the therapeutically effective amount of the drug can reduce the number of cancer cells; reduce the tumor size; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof. See the definition herein of “treating.” To the extent the drug can prevent growth and/or kill existing cancer cells, it can be cytostatic and/or cytotoxic.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

A “chemotherapeutic agent” is a chemical compound useful in the treatment of cancer, regardless of mechanism of action. Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with the disorder, and may also include those who have minimal residual disease, or resistant disease, or replased disease. In certain embodiments, a subject is successfully “treated” for cancer according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

“Polynucleotide” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR*, CO or CH₂ (“formacetal”), in which each R or R is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “vector” means a construct, which is capable of delivering, and expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains. In some embodiments, a polypeptide, peptide, or protein is non-naturally occurring. In some embodiments, a polypeptide, peptide, or protein is purified from other naturally occurring components. In some embodiments, the polypeptide, peptide, or protein is recombinantly produced.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al., Proc. Natl. Acad. Sci. 87:2264-2268, 1990, as modified in Karlin et al., Proc. Natl. Acad. Sci. 90:5873-5877, 1993, and incorporated into the NBLAST and XBLAST programs (Altschul et al., Nucleic Acids Res. 25:3389-3402, 1991). In certain embodiments, Gapped BLAST can be used as described in Altschul et al., Nucleic Acids Res. 25:3389-3402, 1997; BLAST-2, WU-BLAST-2 (Altschul et al., Methods in Enzymology 266:460-480, 1996), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453, 1970) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17, 1989). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489, 1981, to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In certain embodiments, identity exists over a region of the sequences that is at least about 10, about 20, about 40-60 residues in length or any integral value therebetween, or over a longer region than 60-80 residues, at least about 90-100 residues, or the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the invention do not abrogate the binding of the polypeptide or antibody containing the amino acid sequence, to the antigen(s), i.e., the CD123/IL-3Ra to which the polypeptide or antibody binds. Methods of identifying nucleotide and amino acid conservative substitutions which do not eliminate antigen-binding are well-known in the art (see, e.g., Brummell et al., Biochem. 32:1180-1187, 1993; Kobayashi et al., Protein Eng. 12(10):879-884, 1999; and Burks et al., Proc. Natl. Acad. Sci. USA 94:412-417, 1997).

Another aspect of the present invention provides an antibody protein according to the invention linked to a therapeutic agent, wherein said therapeutic agent is a therapeutic agent selected from the group consisting of radioisotopes, toxins, toxoids, pro-drugs and chemotherapeutic agents.

In certain embodiments, the therapeutic agent is linked to the antibody protein via a linker selected from the group of MAG-3 (U.S. Pat. No. 5,082,930 A, EP 0247866 B1 (page 2 lines 55-56-page 3 lines 1-23)); MAG-2 GABA (U.S. Pat. No. 5,681,927 A, EP 0284071 B1 (page 6 lines 9-29)); and N2S2 ((=phenthioate) U.S. Pat. Nos. 4,897,255 A, 5,242,679 A, EP 0188256 B1 (page 2, lines 38-page 3, lines 18)), (Ac)Phe-Lys(Alloc)-PABC-PNP, 6-Maleimidohexanoic acid N-hydroxysuccinimide ester, 6-Quinoxalinecarboxylic acid, 2,3-bis(bromomethyl)-Fmoc-Val-Cit-PAB, Fmoc-Val-Cit-PAB-PNP, Mc-Val-Cit-PABC-PNP, Val-cit-PAB-OH, all herein incorporated by reference.

In certain embodiments, the radioisotope is selected from the group consisting of ¹⁸⁶Rhenium, ¹⁸⁸Rhenium, ¹³¹Iodine, and ⁹⁰Yttrium.

In certain embodiments, the antibody proteins according to the invention are labelled. Such CD44v6- or CD44v9-specific labelled antibody allows for the localization and/or detection of the CD44v6/CD44v9 antigen in vitro and/or in vivo.

A label is defined as a marker that may be directly or indirectly detectable. An indirect marker is defined as a marker that cannot be detected by itself but needs a further directly detectable marker specific for the indirect marker. Preferred labels for practicing the invention are detectable markers. From the large variety of detectable markers, a detectable marker may be selected from the group consisting of enzymes, dyes, radioisotopes, digoxygenin, and biotin.

In certain embodiments, the label is a detectable marker, such as one selected from the group consisting of enzymes, dyes, radioisotopes, digoxygenin, and biotin.

In certain embodiments, antibody proteins according to the invention are conjugated to an imageable agent. A large variety of imageable agents, especially radioisotopes, are available from the state of the art. In certain embodiments, the imageable agent is gamma-emitting isotopes, such as ¹²⁵Iodine. In certain embodiments, the antibody protein has specific activity of from about 0.5 to about 15 mCi/mg, or from about 0.5 to about 14 mCi/mg, or about 1 to about 10 mCi/mg, or about 1 to about 5 mCi/mg, and about 2 to 6 mCi/mg or 1 to 3 mCi/mg.

### 3. Compositions and Pharmaceutical Compositions

The present invention includes a composition (e.g., a pharmaceutical composition) comprising the subject antibodies or antigen-binding fragments thereof, or immuno-conjugates thereof described herein, and a carrier (e.g., a pharmaceutically acceptable carrier). The present invention also includes a composition (e.g., a pharmaceutical composition) comprising the subject antibodies or antigen-binding fragments thereof, or conjugate thereof, and a carrier (a pharmaceutically acceptable carrier), and further comprising a second therapeutic agent. The present compositions are useful for inhibiting abnormal cell growth or treating a proliferative disorder in a mammal (e.g., human), including hematologic cancer, leukemia, or lymphoma.

In particular, the present invention provides pharmaceutical compositions comprising one or more of the CD44v6 or CD44v9-binding agents or immuno-conjugates thereof described herein. In certain embodiments, the pharmaceutical compositions further comprise a pharmaceutically acceptable vehicle. These pharmaceutical compositions find use in inhibiting tumor growth and treating cancer in human patients, including hematologic cancer, leukemia, or lymphoma.

In certain embodiments, formulations are prepared for storage and use by combining a purified antibody, or immuno-conjugate thereof of the present invention with a pharmaceutically acceptable vehicle (e.g. carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and non-ionic surfactants such as TWEEN or polyethylene glycol (PEG).

A pharmaceutically acceptable carrier can contain physiologically acceptable compounds that act, for example, to stabilize or to increase the absorption of an AMPA glutamate receptor agonist, antagonist or modulator. Such physiologically acceptable compounds include, for example, carbohydrates, such as glucose, sucrose or dextrans, antioxidants, such as ascorbic acid or glutathione, chelating agents, low molecular weight proteins or other stabilizers or excipients (see also e.g. Remington's Pharmaceutical Sciences (1990), 18th ed. Mack Publ., Easton). One skilled in the art would know that the choice of a pharmaceutically acceptable carrier, including a physiologically acceptable compound, depends, for example, on the route of administration of the composition.

Suitable pharmaceutically acceptable carriers, diluents, and excipients are generally well known and can be determined by those of ordinary skill in the art as the clinical situation warrants. Examples of suitable carriers, diluents and/or excipients include: (1) Dulbecco's phosphate buffered saline, pH about 7.4, containing or not containing about 1 mg/mL to 25 mg/mL human serum albumin, (2) 0.9% saline (0.9% w/v NaCl), and (3) 5% (w/v) dextrose; and may also contain an antioxidant such as tryptamine and a stabilizing agent such as Tween 20.

The pharmaceutical compositions described herein can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration. In some particular embodiments, the administration is intravenous. The pharmaceutical compositions described herein can also be used in vitro or in ex vivo.

In an animal or human body, it can prove advantageous to apply the pharmaceutical compositions as described above via an intravenous or other route, e.g. systemically, locally or topically to the tissue or organ of interest, depending on the type and origin of the disease or problem treated, e.g. a tumor. For example, a systemic mode of action is desired when different organs or organ systems are in need of treatment as in e.g. systemic autoimmune diseases, or allergies, or transplantations of foreign organs or tissues, or tumors that are diffuse or difficult to localise. A local mode of action would be considered when only local manifestations of neoplastic or immunologic action are expected, such as, for example local tumors.

The pharmaceutical compositions comprising antibody proteins of the present invention may be applied by different routes of application known to the expert, notably intravenous injection or direct injection into target tissues. For systemic application, the intravenous, intravascular, intramuscular, intraarterial, intraperitoneal, oral, or intrathecal routes are preferred. A more local application can be effected subcutaneously, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, pharmaceutical antibody compositions may be administered once or several times, also intermittently, for instance on a daily basis for several days, weeks or months and in different dosages.

For preparing suitable pharmaceutical compositions comprising antibody preparations for the applications described above, one may use known injectable, physiologically acceptable sterile solutions. For preparing a ready-to-use solution for parenteral injection or infusion, aqueous isotonic solutions, such as e.g. saline or corresponding plasma protein solutions are readily available. The pharmaceutical compositions may be present as lyophylisates or dry preparations, which can be reconstituted with a known injectable solution directly before use under sterile conditions, e.g. as a kit of parts. The final preparation of the antibody compositions of the present invention are prepared for injection, infusion or perfusion by mixing purified antibodies according to the invention with a sterile physiologically acceptable solution, that may be supplemented with known carrier substances or/and additives (e.g. serum albumin, dextrose, sodium bisulfite, EDTA).

The amount of the antibody applied depends on the nature of the disease. In cancer patients, the applied dose of a “naked” antibody which is comprised in the pharmaceutical composition according to the invention may be between 0.1 and 100 mg/m², between 5 and 50 mg/m² per application, 10 mg/m² to about 40 mg/m², 10 mg/m² to about 30 mg/m², also 20 mg/m² to about 30 mg/², and about 25 mg/m² body surface area. An antibody protein dose of about 50 mg/m² body surface area can also be used.

The dose of radioactivity applied to the patient per administration has to be high enough to be effective, but must be below the dose limiting toxicity (DLT). For pharmaceutical compositions comprising radiolabeled antibodies, e.g. with ¹⁸⁶Rhenium, the maximally tolerated dose (MTD) has to be determined which must not be exceeded in therapeutic settings. Application of radiolabeled antibody to cancer patients may then be carried out by repeated (monthly or weekly) intravenous infusion of a dose which is below the MTD (See e.g. Welt et al. (1994) J. Clin. Oncol. 12: 1193-1203). Multiple administrations are preferred, generally at weekly intervals; however, radiolabelled materials should be administered at longer intervals, i.e., 4-24 weeks apart, preferable 12-20 weeks apart. The artisan may choose, however, to divide the administration into two or more applications, which may be applied shortly after each other, or at some other predetermined interval ranging, e.g. from 1 day to 1 week.

Furthermore, the applied radioactivity dose will be in accordance with the guidelines outlined below. In general, the radioactivity dose per administration will be between 30 and 75 mCi/m² body surface area (BSA). Thus, the amount of radiolabelled antibody in the pharmaceutical composition according to the invention, labelled with ¹⁸⁶Rhenium, ¹⁸⁸Rhenium, ^{99m}Technetium, ¹³³Iodine, or ⁹⁰Yttrium, preferably labelled with ¹⁸⁶Rhenium, to be applied to a patient is 10, 20, 30, 40, 50 or 60 mCi/m², preferably 50 mCi/m². In one embodiment, the invention relates to a pharmaceutical composition, wherein the dose of said radiolabelled antibody according to the invention is MTD, 50 mCi/m².

In certain embodiments, the pharmaceutical composition according to the invention further comprising one or more radioprotectants selected from the group of ascorbic acid, gentisic acid, reductic acid, erythrorbic acid, p-aninobenzoic acid, 4hydroxybenzoic acid, nicotinic acid, nicotinamide, 2-5-dihydroxy-1,4-benzenedisulfonic acid, povidone, inositol, and/or citrate. In certain embodiments, the radioprotectant is ascorbic acid.

An antibody or immunoconjugate of the invention can be combined in a pharmaceutical combination formulation, or dosing regimen as combination therapy, with a second compound, such as one that is known to be effective in treating a disease or disorder of interest. In some embodiments, the second compound is a anti-cancer agent. In some embodiments, the methods encompass administration of the second compound and an immunoconjugate of the invention that results in a better efficacy as compared to administration of the immunoconjugate alone. The second compound can be administered via any number of ways, including for example, topical, pulmonary, oral, parenteral, or intracranial administration. In some embodiments, the administration is oral. In some embodiments, the administration is intravenous. In some embodiments, the administration is both oral and intravenous.

An antibody or immunoconjugate can also be combined in a pharmaceutical combination formulation, or dosing regimen as combination therapy, with an analgesic, or other medications.

An antibody or immunoconjugate can be combined in a pharmaceutical combination formulation, or dosing regimen as combination therapy, with a second compound having anti-cancer properties. The second compound of the pharmaceutical combination formulation or dosing regimen can have complementary activities to the ADC of the combination such that they do not adversely affect each other. Pharmaceutical compositions comprising the CD44v6- or CD44v9-binding agent and the second anti-cancer agent are also provided.

In certain embodiments, the therapeutically effective amount of the subject antibodies or antigen-binding fragments thereof, or immuno-conjugates described herein, or a composition thereof, alone or in combination with a second therapeutic agent, preferentially inhibits the proliferation of leukemic stem cells (LSCs), leukemia progenitors (LPs), and/or leukemic blasts, over normal hematopoietic stem cells (HSCs). In certain embodiments, IC₅₀ value or the half maximum concentration of the above subject agents to inhibit the proliferation of leukemic stem cells (LSCs), leukemia progenitors (LPs), and/or leukemic blasts, is at least 10-, 20-, 30-, 40-, 50-, 60-, 70-, 80-, 90-, 100-, 150-, 300-, 500-fold or more lower than that for the normal hematopoietic stem cells (HSCs).

### 4. Method of Treatment

The present invention includes a method of inhibiting abnormal cell growth or treating a proliferative disorder in a mammal (e.g., human) comprising administering to said mammal a therapeutically effective amount of the subject antibodies or antigen-binding fragments thereof, or immuno-conjugates described herein, or a composition thereof, alone or in combination with a second therapeutic agent.

Another aspect of the present invention is the use of an antibody protein according to the invention in the manufacture of a medicament for treatment of cancer. Another aspect of the present invention relates to the use of antibody proteins according to the invention conjugated to a therapeutic agent as described above for the treatment of cancer. Cancer includes any disease associated with malignant growth such as solid tumors, sarcomas and leukemias. A necessary precondition for such diseases is the expression of CD44v6 or CD44v9.

The present invention also provides a method for inducing cell death in selected cell populations comprising contacting target cells or tissue containing target cells with an effective amount of the subject antibodies or antigen-binding fragments thereof, or immuno-conjugates of the present invention. The target cells are cells to which the cell-binding agent of the conjugates can bind.

The method of the invention for inducing cell death in selected cell populations, for inhibiting cell growth, and/or for treating cancer, can be practiced in vitro, in vivo, or ex vivo. For clinical in vivo use, the cytotoxic compounds or conjugates of the invention will be supplied as a solution or a lyophilized powder that are tested for sterility and for endotoxin levels.

In certain embodiments, the abnormal cell growth or proliferative disorder in a mammal is a disease or condition associated with or characterized by the expression of CD44v6 or CD44v9, such as cancer.

For example, the cancer may be selected from the group consisting of: epithelial carcinomas including breast, lung, liver, colorectal, head and neck, esophageal, pancreatic, ovarian, bladder, gastric, skin, endometrial, ovarian, testicular, esophageal, prostatic and renal origin; bone and soft-tissue sarcomas including osteosarcoma, chondrosarcoma, fibrosarcoma, malignant fibrous histiocytoma (MFH), and leiomyo sarcoma; hematopoietic malignancies including lymphomas and leukemias; neuroectodermal tumors including peripheral nerve tumors, astrocytomas and melanomas, and mesotheliomas.

Cancer according to the invention may also include, and is not limited to: 1) The treatment of epithelial carcinomas including breast, lung, liver, colorectal, head and neck, esophageal, pancreatic, ovarian, bladder, gastric, skin, endometrial, ovarian, testicular, esophageal, prostatic and renal origin; 2) Bone and soft-tissue sarcomas: Osteosarcoma, chondrosarcoma, fibrosarcoma, malignant fibrous histiocytoma (MFH), leiomyosarcoma; 3) Hematopoietic malignancies: Hodgkin's and non-Hodgkin's lymphomas, leukemias; 4) Neuroectodermal tumors: Peripheral nerve tumors, astrocytomas, melanomas; 5) Mesotheliomas.

Examples for cancerous disease states associated with solid tumors include, but are not limited to: colorectal cancer, non-small cell lung cancer, breast cancer, head and neck cancer, ovarian cancer, lung cancer, bladder cancer, pancreatic cancer and metastatic cancers of the brain

In certain embodiments, the cancer has at least one negative prognostic factor.

Another aspect of the invention relates to the use of an antibody protein according to the invention as defined supra in the manufacture of a medicament for treatment of cancer, wherein the amount of antibody protein per application is between 0.1 and 100 mg/m², between 5 and 50 mg/m², 10 mg/m² to about 40 mg/m², 10 mg/m² to about 30 mg/m², or 20 mg/m² to about 30 mg/m², or about 25 mg/m² body surface area, or about 50 mg/m² body surface area.

In certain embodiments, an antibody protein conjugated to a radioisotope according to the invention as defined supra is used in the manufacture of a medicament for treatment of cancer, wherein the radioactivity dose per administration is between 30 and 75 mCi/m² body surface area (BSA). In certain embodiments, the antibody protein according to the invention is radiolabelled with ¹⁸⁶Rhenium, ¹⁸⁸Rhenium, ^{99m}Technetium, ¹³¹Iodine, or ⁹⁰Yttrium, such as ¹⁸⁶Rhenium. In yet another embodiment, the invention relates to the use of an antibody protein conjugated to a radioisotope according to the invention as defined supra in the manufacture of a medicament for treatment of cancer, wherein to antibody dose is 10, 20, 30, 40, 50 or 60 mCi/m², or 50 mCi/m².

In certain embodiments, an antibody protein conjugated to a radioisotope according to the invention as defined supra is used in the manufacture of a medicament for treatment of cancer, wherein the antibody protein has specific activity of from about 0.5 to about 15 mCi/mg, or from about 0.5 to about 14 mCi/mg, preferably about 1 to about 10 mCi/mg, preferably about 1 to about 5 mCi/mg, and most preferably 2 to 6 mCi/mg or 1 to 3 mCi/mg.

Preferred also is the use of an antibody protein conjugated to a radioisotope according to the invention as defined supra in the manufacture of a medicament for treatment of cancer, wherein said antibody or antibody derivative is in an aqueous solution at pH of from about 7 to about 8, and at a concentration of from about 0.5 to about 2.0 mg/ml.

The invention further relates to a method of cancer treatment, wherein an antibody protein according to the invention is administered once to several times to an individual in need thereof, said antibody protein selectively binds to CD44v6 or CD44v9, destroys tumor cells via the therapeutic agent linked to the antibody protein and the therapeutic success is monitored. Said antibody protein may be present as naked/unmodified antibody protein, modified antibody protein, such as e.g. fusion protein, or antibody protein conjugated to a therapeutic agent, which comprises contacting the tumor with an effective amount of said antibodies. The method of treating tumors as described above may be effective in vitro or in vivo. Cancer is any cancer as described above.

Cancer therapies and their dosages, routes of administration and recommended usage are known in the art and have been described in such literature as the Physician's Desk Reference (PDR). The PDR discloses dosages of the agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician. The contents of the PDR are expressly incorporated herein in its entirety by reference. One of skill in the art can review the PDR, using one or more of the following parameters, to determine dosing regimen and dosages of the chemotherapeutic agents and conjugates that can be used in accordance with the teachings of this invention. These parameters include: Comprehensive index; Manufacturer; Products (by company's or trademarked drug name); Category index; Generic/chemical index (non-trademark common drug names); Color images of medications; Product information, consistent with FDA labeling; Chemical information; Function/action; Indications & Contraindications; Trial research, side effects, warnings.

The amount of the antibody applied depends on the nature of the disease. In cancer patients, the applied dose of a “naked” antibody may be between 0.1 and 100 mg/m², between 5 and 50 mg/m² per application, 10 mg/m² to about 40 mg/m², 10 mg/m² to about 30 mg/m, also 20 mg/m² to about 30 mg/m², and about 25 mg/m² body surface area, or about 50 mg/m² body surface area.

The dose of radioactivity applied to the patient per administration has be high enough to be effecfive, but must be below the dose limiting toxicity (DLT). For radiolabeled antibodies, e.g. with ¹⁸⁶Rhenium, the maximally tolerated dose (MTD) has to be determined which must not be exceeded in therapeutic settings. Application of radiolabeled antibody to cancer patients may then be carried out by repeated (monthly or weekly) intravenous infusion of a dose which is below the MTD (See e.g. Welt et al. (1994) J. Clin. Oncol. 12: 1193-1203). Multiple administrations are preferred, generally at weekly intervals; however, radiolabelled materials should be administered at longer intervals, i.e., 4-24 weeks apart, or 12-20 weeks apart. The artisan may choose, however, to divide the administration into two or more applications, which may be applied shortly after each other, or at some other predetermined interval ranging, e.g. from 1 day to 1 week.

Also provided a method of cancer treatment according to the invention (see above), wherein the antibody protein conjugated to a radioisotope according to the invention as defined supra has specific activity of from about 0.5 to about 15 mCi/mg, or from about 0.5 to about 14 mCi/mg, preferably about 1 to about 10 mCi/mg, preferably about 1 to about 5 mCi/mg, and most preferably 2 to 6 mCi/mg or 1 to 3 mCi/mg.

Also provided is a method of cancer treatment according to the invention (see above), wherein the antibody protein conjugated to a radioisotope according to the invention as defined supra is in an aqueous solution at pH of from about 7 to about 8, and at a concentration of from about 0.5 to about 2.0 mg/ml.

In certain embodiments, the cancer is colorectal cancers, non-small cell lung cancers, breast cancers, head and neck cancer, ovarian cancers, lung cancers, bladder cancers, pancreatic cancers or metastatic cancers of the brain.

The method of the invention also provides in vitro method to kill cells, such as cancer cells. Examples of in vitro uses include treatments of autologous bone marrow prior to their transplant into the same patient in order to kill diseased or malignant cells: treatments of bone marrow prior to their transplantation in order to kill competent T cells and prevent graft-versus-host-disease (GVHD); treatments of cell cultures in order to kill all cells except for desired variants that do not express the target antigen; or to kill variants that express undesired antigen.

The conditions of non-clinical in vitro use are readily determined by one of ordinary skill in the art.

Examples of clinical ex vivo use are to remove tumor cells or lymphoid cells from bone marrow prior to autologous transplantation in cancer treatment or in treatment of autoimmune disease, or to remove T cells and other lymphoid cells from autologous or allogenic bone marrow or tissue prior to transplant in order to prevent GVHD. Treatment can be carried out as follows. Bone marrow is harvested from the patient or other individual and then incubated in medium containing serum to which is added the cytotoxic agent of the invention, concentrations range from about 10 μM to 1 pM, for about 30 minutes to about 48 hours at about 37° C. The exact conditions of concentration and time of incubation, i.e., the dose, are readily determined by one of ordinary skill in the art. After incubation the bone marrow cells are washed with medium containing serum and returned to the patient intravenously according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

### 5. Nucleic Acid

A further aspect of the present invention is a nucleic acid, characterised in that it codes for an antibody or protein according to the invention. Said nucleic acid may be RNA or preferably DNA. Said DNA molecule may be chemically synthesized. First, suitable oligonucleotides can be synthesized with methods known in the art (e.g. Gait, M. J., 1984, Oligonucleotide Synthesis. A Practical Approach. IRL Press, Oxford, UK), which can be used to produce a synthetic gene. Methods to generate synthetic genes are known in the art (e.g. Stemmer et al. 1995, Single-step assembly of a gene and entire plasmid from large numbers of oligodeoxyribonucleotides, Gene 164(1): 49-53; Ye et al. 1992, Gene synthesis and expression in E. coli for pump, a human matrix metalloproteinase, Biochem Biophys Res Commun 186(1):143-9; Hayden et Mandecki 1988, Gene synthesis by serial cloning of oligonucleotides, DNA 7(8): 571-7). These methods can be used to synthesize any DNA molecule disclosed in the present application.

The nucleic acid according to the invention may contain 5′ or 3′ or 5′ and 3′ untranslated regions. The nucleic acid according to the invention may contain other untranslated regions upstream and/or downstream. The untranslated region may contain a regulatory element, such as e.g. a transcription initiation unit (promoter) or enhancer. Said promoter may, for example, be a constitutive, inducible or development-controlled promoter. In certain embodiments, and without ruling out other known promoters, the constitutive promoters of the human Cytomegalovirus (CMV) and Rous sarcoma virus (RSV), as well as the Simian virus 40 (SV40) and Herpes simplex promoter. Inducible promoters according to the invention comprise antibiotic-resistance promoters, heat-shock promoters, hormone-inducible “Mammary tumour virus promoter” and the metallothioneine promoter. The nucleic acid according to the invention may codes for a fragment of the antibody protein according to the invention. This refers to part of the polypeptide according to the invention.

### 6. Vector

Another important aspect of the present invention is a recombinant DNA vector, characterised in that it contains a nucleic acid according to the invention. Examples are viral vectors such as e.g. Vaccinia, Semliki-Forest-Virus and Adenovirus. Vectors for use in COS-cells have the SV40 origin of replication and make it possible to achieve high copy numbers of the plasmids. Vectors for use in insect cells are, for example, E. coli transfer vectors and contain e.g. the DNA coding for polyhedrin as promoter.

Another aspect of the present invention is a recombinant DNA vector according to the invention, characterized in that it is an expression vector.

Another aspect of the present invention is a recombinant DNA vector according to the invention, characterized in that it is vector pAD-CMV or a functional derivative thereof. Such derivatives are e.g. pAD-CMV1, pAD-CMV19 or pAD-CMV25.

The vector may be the ones disclosed in U.S. Pat. Nos. 5,648,267 A or 5,733,779 A comprising a nucleotide sequence according to the invention. Another aspect of the present invention is a recombinant DNA vector according to the invention, characterized in that it is vector N5KG1Val or a derivative thereof.

### 7. Cell or Host Cell

Another aspect is a host, characterised in that it contains a vector according to the invention.

Another aspect is a host according to the invention, characterized that it is a eukaryotic host cell. The eukaryotic host cells according to the invention include fungi, such as e.g. Pichia pastoris, Saccharomyces cerevisiae, Schizosaccharomyces, Trichoderma, insect cells (e.g. from Spodoptera frugiperda Sf-9, with a Baculovirus expression system), plant cells, e.g. from Nicotiana tabacum, mammalian cells, e.g. COS cells, BHK, CHO or myeloma cells.

In descendants of the cells of the immune system in which antibody proteins are also formed in our body, the antibody proteins according to the invention are particularly well folded and glycosylated. Mammalian host cells, preferably CHO or COS cells are preferred, e.g. a CHO DG44 (Urlaub and Chasin, Proc. Natl. Acad. Sci. U.S.A. 77(7): 4216-20 (1980)), or CHO-K1 (ATCC CCL-61) cells. Thus, another aspect is a host according to the invention according to the invention, characterised in that it is a BHK, CHO or COS cell, most preferred CHO DG44 or CHO-K1 (ATCC CCL-61) cells.

In certain embodiments, the host is a bacteriophage.

In certain embodiments, the host is a prokaryotic host cell. Examples of prokaryotic host cells are Escherichia coli, Bacillus subtilis, Streptomyces or Proteus mirabilis.

The invention further relates to a process for preparing an antibody protein according to the invention, characterized in that it comprises the following steps: a host according to the invention is cultivated under conditions in which said antibody protein is expressed by said host cell and said antibody protein is isolated. The antibody according to the invention may be produced as follows. Nucleic acid molecules coding for the light chain and the heavy chain may be synthesised chemically and enzymatically by standard methods. First, suitable oligonucleotides can be synthesized with methods known in the art (details supra). Methods to generate synthetic genes from oligonucleotides are known in the art (details supra). These nucleic acid molecules encoding the antibody heavy and light chains may be cloned into an expression vector (either both chains in one vector molecule, or each chain into a separate vector molecule), which then is introduced into a host cell. The host cell may be a mammalian host cell (details supra), e.g. a COS, CHO (Chinese Hamster Ovary), or BHK cell. The host cell then is cultured in a suitable culture medium under conditions where the antibody is produced, and the antibody is then isolated from the culture according to standard procedures. Procedures for production of antibodies from recombinant DNA in host cells and respective expression vectors are well-known in the art (see e.g. WO 94/11523, WO 97/9351, EP 0481790).

The invention also relates to a process, wherein the host is a mammalian cell, preferably a CHO or COS cell.

In certain embodiments, the host cell is co-transfected with two plasmids which carry the expression units for the light or the heavy chain.

### EXAMPLES

The following examples serve to further illustrate the present invention; but the same should not be construed as limiting the scope of the invention disclosed herein.

### Example 1 Live-Cell MabArray Isolation of the Anti-CD44v6 Monoclonal Antibody mAb119, and the Anti-CD44v9 Monoclonal Antibody mAb116

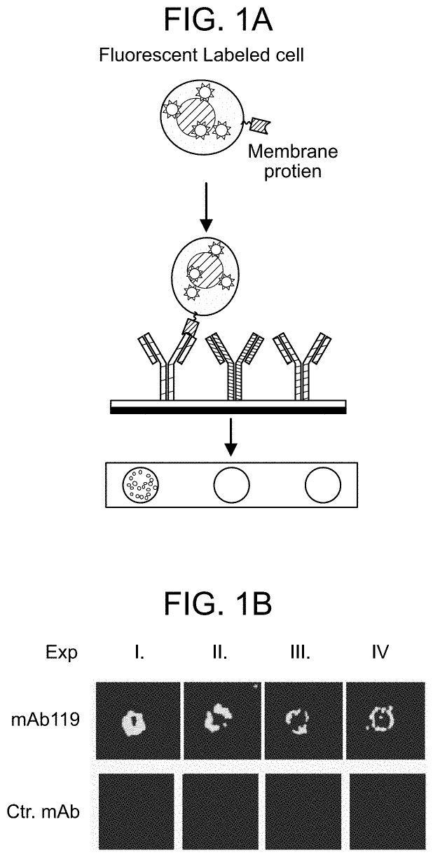

As shown in FIG. 1A, about 6×10⁴ different monoclonal antibodies (mAbs) were printed onto 4 glass aldehyde chips (75×25 mm) using Arrayjet printer to generate MabArray. The MabArray chips were then blocked with 10% BSA overnight, before the experiments were performed. Live lung cancer cell line PC9 cells were labeled with a green fluorescent nucleic acid stain SYTO14 (ThermoFisher Scientific), and incubated with the chips at a density of 1×10⁷ cells/mL in PBS for 1 hour. MabArray chips were then washed with PBS gently and scanned with Genepix scanner.

FIG. 1B shows images of mAb119 and control mAb in 4 independent PC9 live cell MabArray experiments. Live PC9 cells were captured by mAb119 on MabArray chips.

### Example 2 the mAb119 Antigen is Expressed on the Surface of PC9 Cells, and is Internalized by PC9 Cells

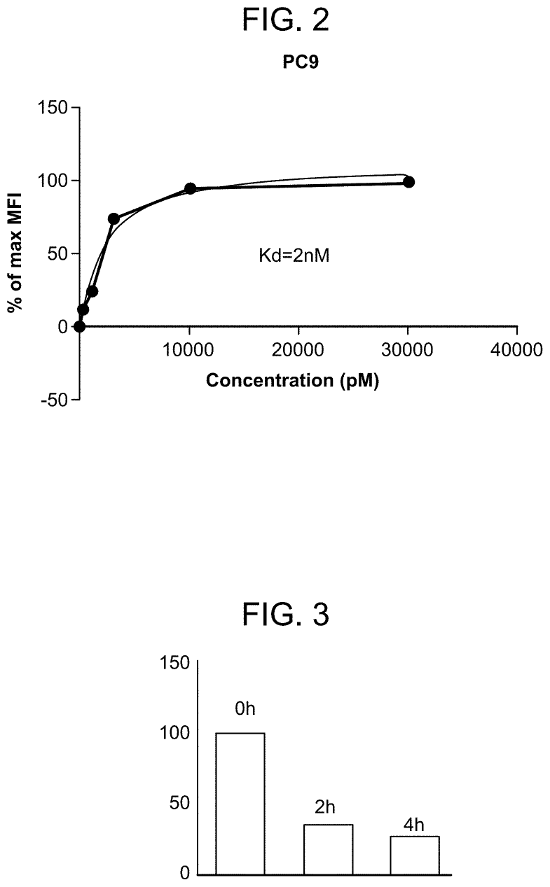

FIG. 2 shows results of FACs analysis of mAb119 on PC9 cells. PC9 FACS titration of mAb119 was performed by incubating PC9 cells with a serial dilution (30000 pM to 0.1 pM, 3 fold serial dilution) of mAb119 for 30 min on ice, before the cells were stained with Alexa488-conjugated anti-mouse IgG (Jackson lab) for 30 min. MFI was analyzed using BD C6. Affinity K_{D} was determined to be about 2 nM.

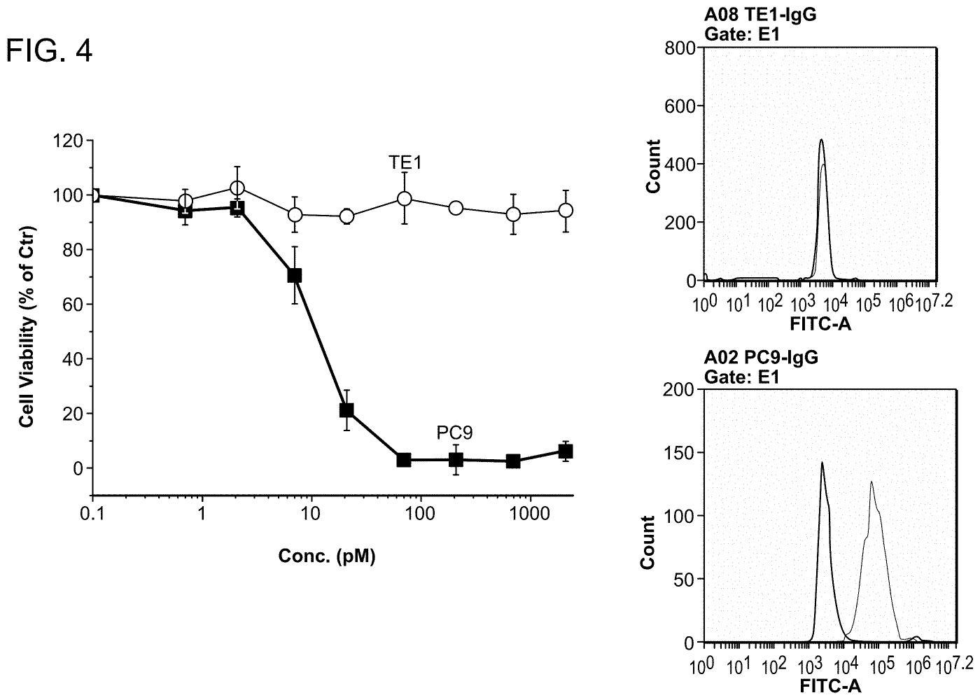

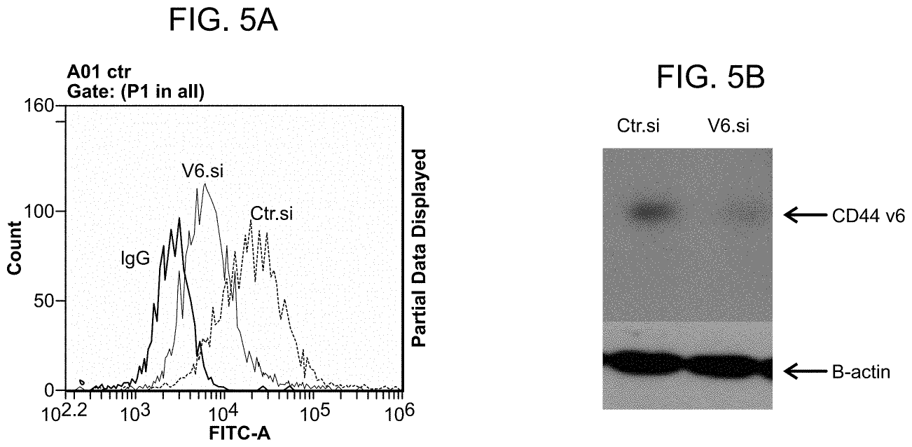

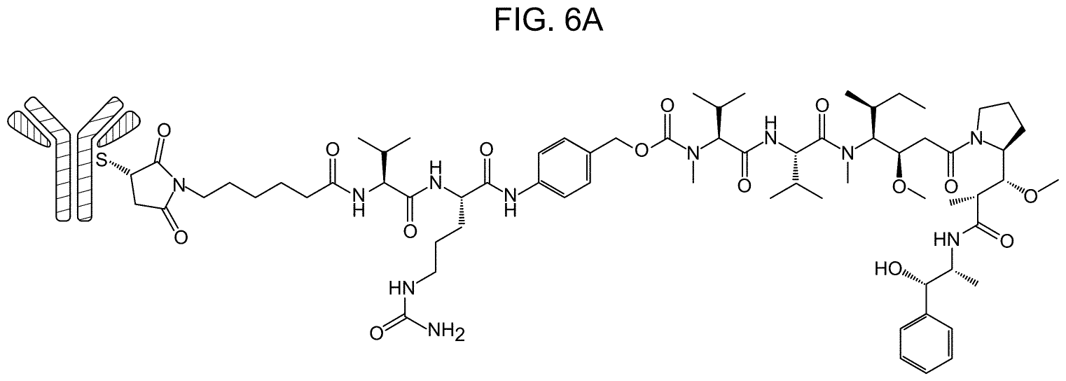

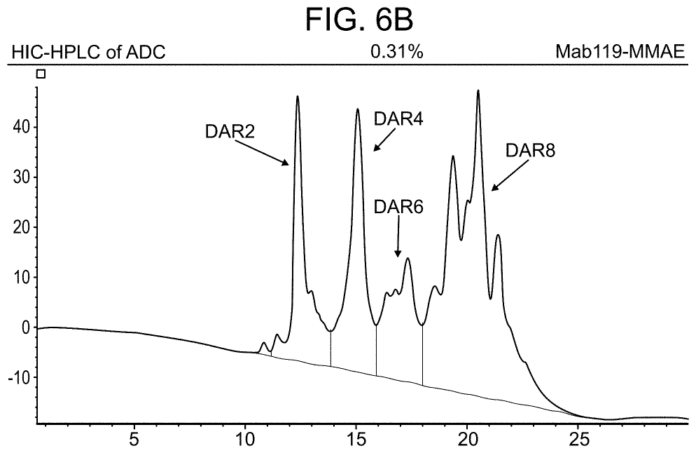

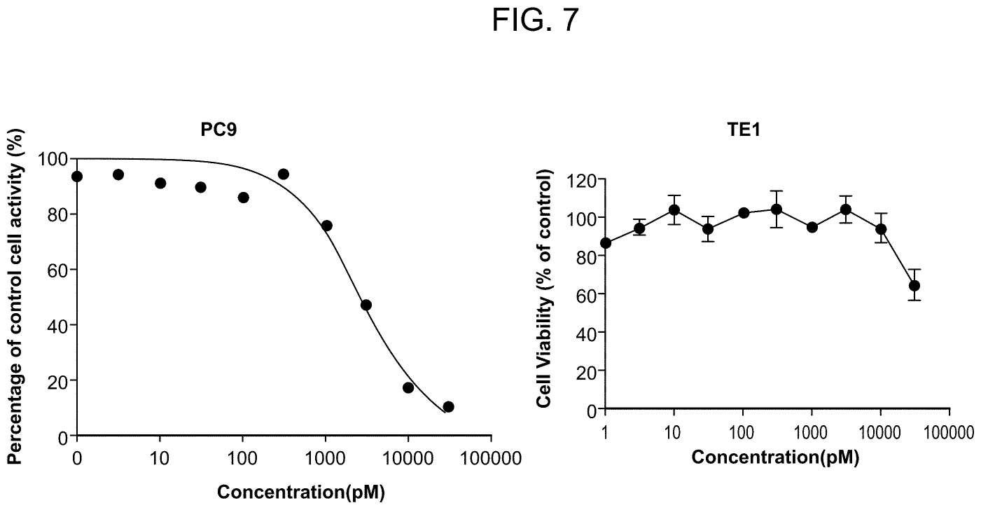

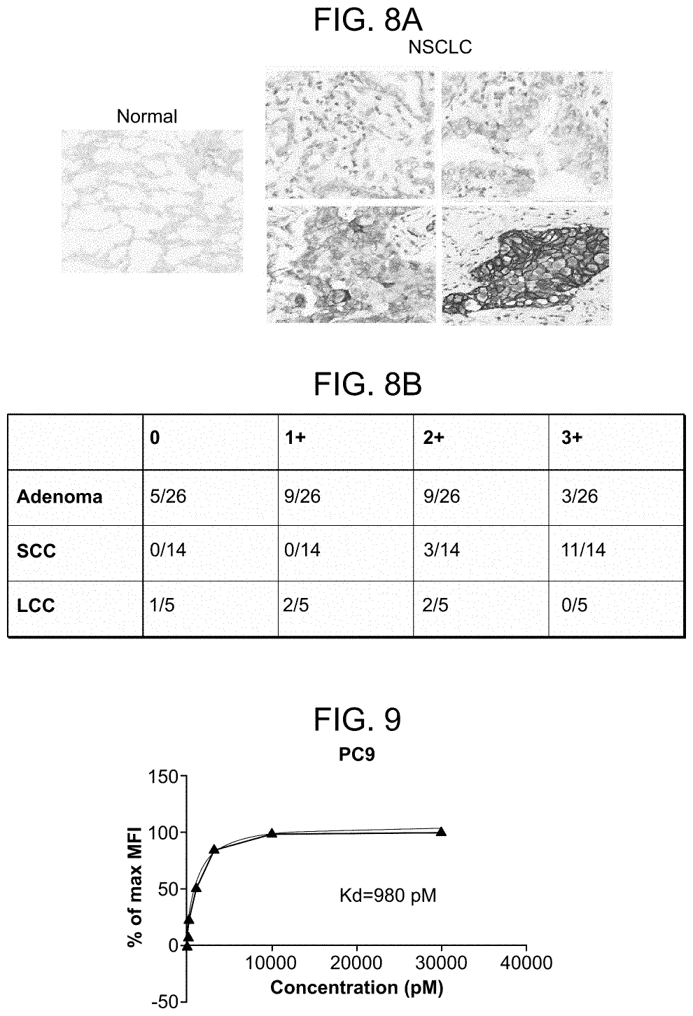

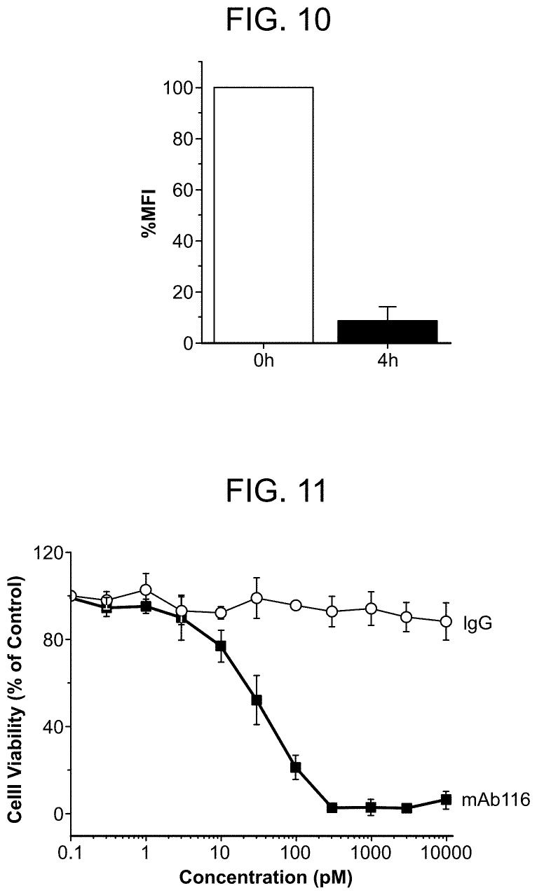

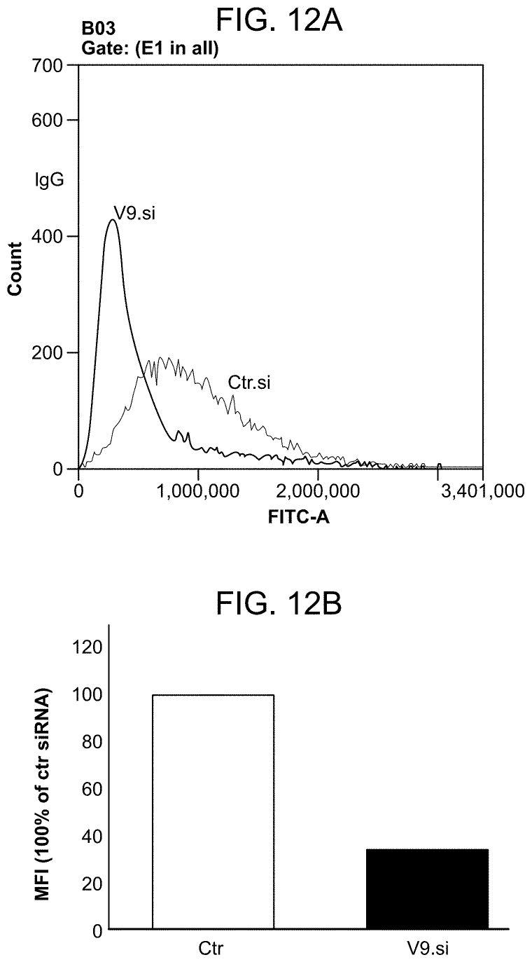

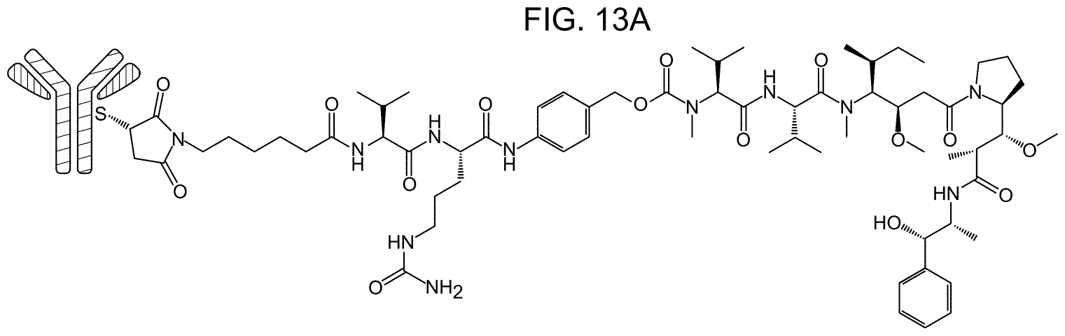

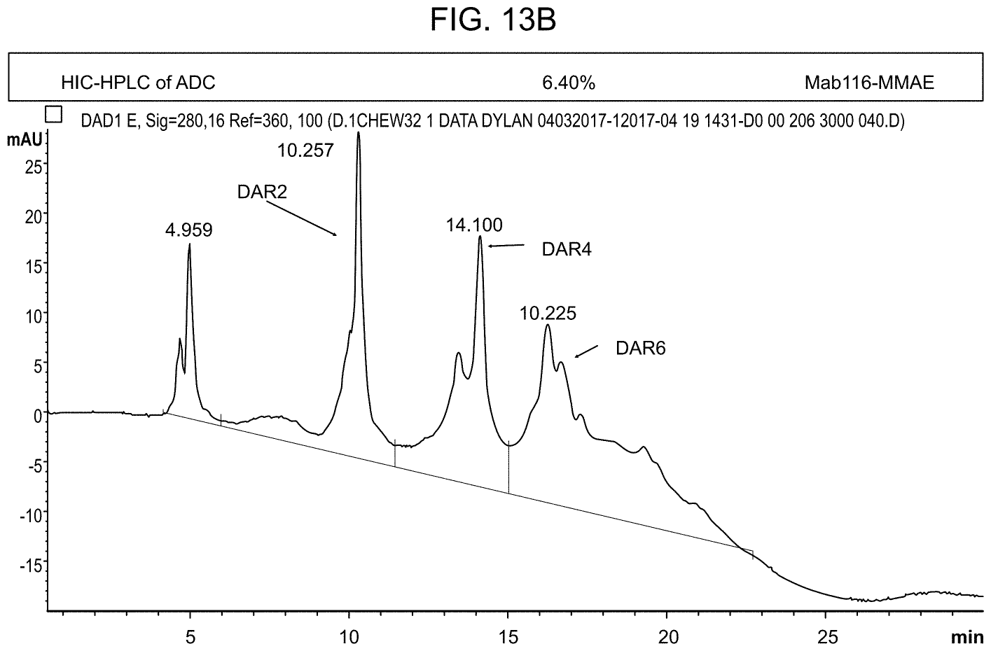

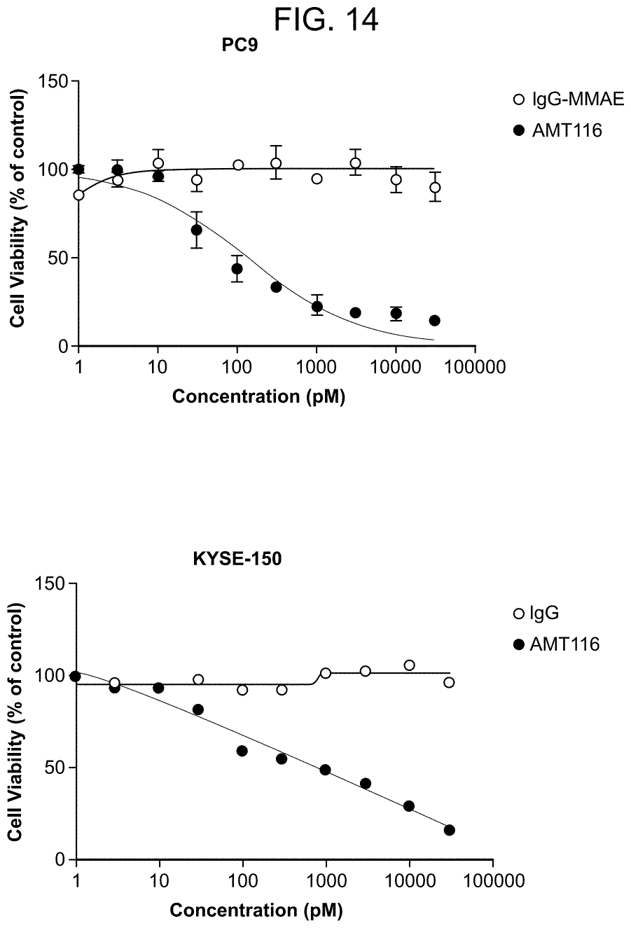

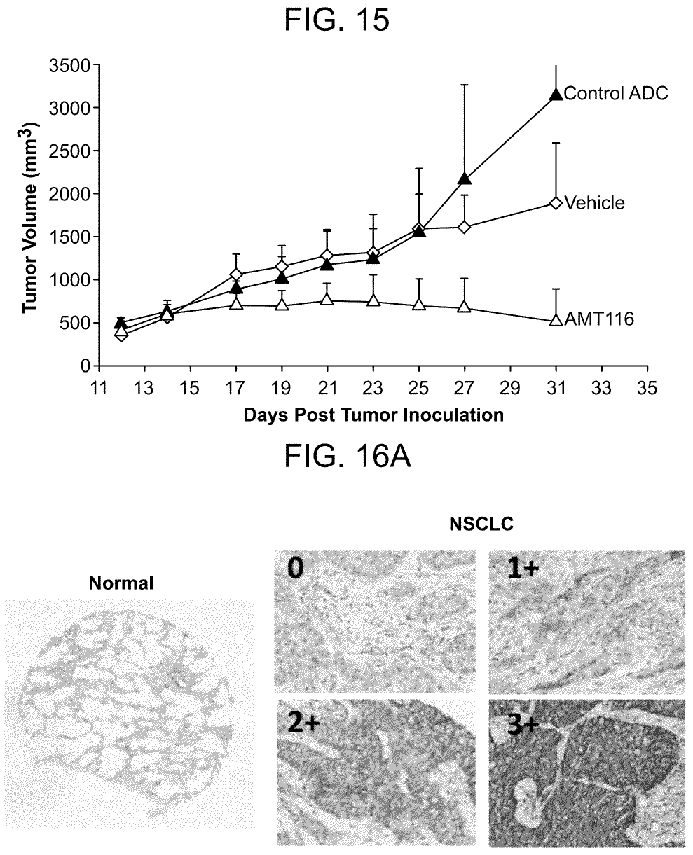

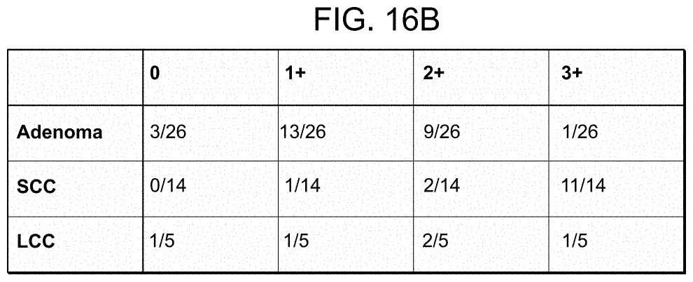

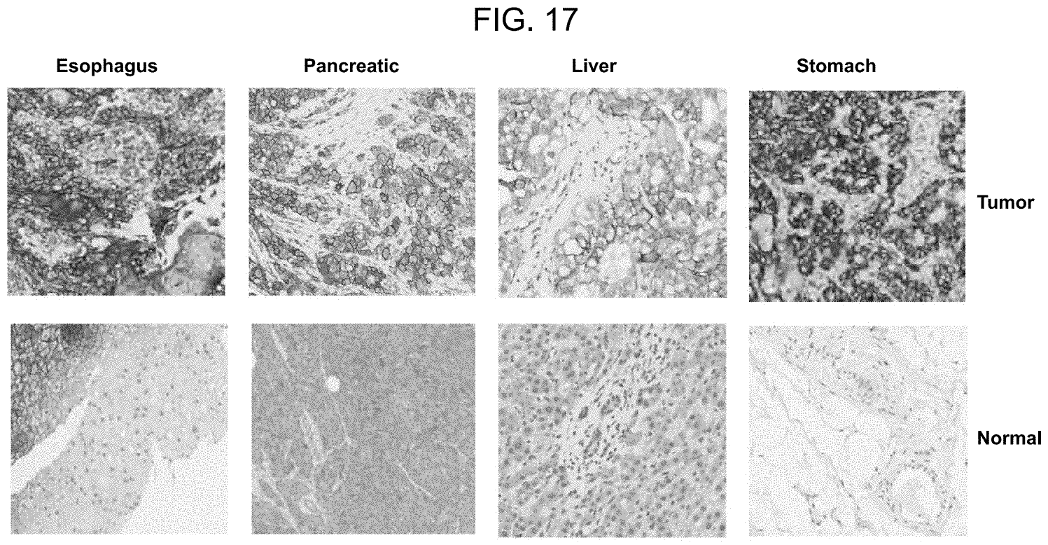

FIG. 3 shows that PC9 cells internalized bound mAb119. Live PC cells were cultured on coverslips, and were incubated with 10 m/mL mAb119 for 1 hr on ice, before the cells were washed 3 times with PBS. Cells were then cultured at 37° C. for 0 hr, 2 hr, or 4 hr, before fixation with 4% PFA before detected with FITC conjugated secondary antibody by FACs. PC9 cells were then co-stained by mAb119 (labeled by a green fluorescent dye Alexa488) and anti-LAMP1 (labeled by a red fluorescent dye Alexa595). Specifically, PC9 cells were permeablized with 0.1% Triton X and incubated with mAb119 and Rabbit anti-LAMP1 antibody (1:200, Abcam) and mAb119 for 1 hr. Antibodies were then labeled with Alexa488 conjugated anti-Mouse antibody and Alexa595 conjugated anti-Rabbit antibody, respectively. Lysosomal-associated membrane protein 1 (LAMP1) is a glycoprotein primarily expressed across lysosomal membranes. Colocalization of mAb119 and anti-LAMP1 signals results in yellow signal that indicates internalization of mAb119 by PC9 cells to the lysosomal compartment. mAb119 was first observed on the cell surface without any co-localization with LAMP1 at 0 hr. Colocalization of mAb119 and LAMP1 was observed at 2 hr and 4 hr.

FACs analysis based on surface fluorescence shows mAb119 internalization on PC9 cells (data not shown). Specifically, live PC9 cells were incubated with 10 μg/mL mAb119 for 0.5 hr on ice before washed 3 times with PBS. Cells were then cultured at 37° C. for 0 hr, 2 hr, or 4 hr, before fixation with 4% PFA. Cells were then stained with Alexa488 conjugated anti-Mouse antibody and analyzed with FACs by calculating surface MFI. Surface MFI, which represented surface localization of mAb119, reduced by 70% and 80% after 2 hr and 4 hr incubation at 37° C., respectively. Shown is the quantification of the FACs data is expressed as the mean percent internalization±SEM (n=3) in PC9 cells. The vertical axis represents the relative surface florescence (%). The data shows that mAb119 could bind membrane antigen and be internalized in PC9 cells.

### Example 3 Indirect Cytotoxicity of mAb119 is Antigen Expression-Dependent

FIG. 4 shows that the indirect cytotoxicity of mAb119 is antigen expression-dependent. PC9 or TE1 cells were cultured in 96-well plate at 2000 cells/well confluence overnight. Cells were treated with serial dilution of mAb119 together with 2 μg/mL MMAE-conjugated goat anti-mouse IgG antibody for 72 hrs. Cell number was then calculated by CCK8 (dojindo). Different cytotoxicity was observed in TE1 and PC9 cells. The antibody cocktail inhibited PC9 growth with an IC₅₀ of 18 pM, while the same antibody cocktail did not inhibit TE1 cell growth. Shown is the representative data derived from TE1 and PC9 cells, expressed as the mean percent growth inhibition±SEM (n=3).

The expression of mAb119 antigen in the two cell lines were also determined by FACs. The side insert panels show FACs analysis of TE1 (top panel) and PC9 (bottom panel) labeled by mAb119. The results suggest that PC9 cells, but not TE1 cells, express mAb119 antigen. Thus the indirect cytotoxicity was positively correlated with antigen expression.

### Example 4 mAb119 Targets Human CD44 v6 Exon

FIGS. 5A and 5B show that mAb119 targets human CD44 v6 exon. PC9 was transfected with a mixture of 4 different siRNAs targeting human CD44 v6 epitope or control siRNA for 48 hrs. Transfected cells were then either stained with mAb119 and analyzed by FACs, or the total protein was extracted and the abundance of mAb119 antigen was evaluated by Western blotting. Knockdown of CD44v6 decreased mAb119 surface staining intensity in FACs (FIG. 5A, FACs data showing that CD44v6 siRNA (V6.si) inhibits surface signal of mAb119 (representative of n=3)). Knockdown of CD44v6 also decreased protein expression level of the mAb119 antigen (FIG. 5B, Western blotting data showing that the CD44v6 siRNA (V6.si) inhibits protein expression of mAb119 antigen (representative of n=3)). The data suggests that mAb119 targets CD44v6.

An antibody-drug-conjugate was prepared using the mAb119 as the antigen-binding moiety. FIG. 6A shows a schematic drawing of the structure of the mAb119-ADC (AMT119), in which mAb119 was conjugated with MC-vc-PAB-MMAE. HPLC-HIC (Hydrophobic Interaction Chromatography) of AMT119 shows that the average drug-antibody ratio (DAR) was about 6. FIG. 6B.

FIG. 7 shows cytotoxicity of AMT119 in PC9 and TE1 cells. Graphs are representative data derived from PC9 and TE1 cells showing the mean percent growth inhibition±SEM of AMT119 (n=3). The IC₅₀ values were 2,600 pM and 39,000 pM in PC9 and TE1 cells, respectively. The difference was consistent with the different expression levels of CD44v6 in the two cell lines (see FIG. 4).

### Example 5 Expression of CD44v6 in Human Non-Small-Cell Lung Cancer

FIGS. 8A and 8B show expression of CD44v6 in human non-small-cell lung cancer (NSCLC, right panels of FIG. 8A) and normal lung tissues (left panel of FIG. 8A). IHC (immunohistochemistry) detection of CD44v6 protein using mAb119 antibody is shown from a series of normal and cancer tissues, showing that CD44v6 was up-regulated in a tumor-specific manner. Photomicrograph images depict tumor tissues representative of 0, 1+, 2+ and 3+ staining intensity (right panels of FIG. 8A). FIG. 8B shows prevalence of CD44v6 in different subtypes of NSCLC. SCC, Squamous cell carcinoma; LCC, Large cell carcinoma.

### Example 6 the mAb116 Antigen is Expressed on the Surface of PC9 Cells, and is Internalized by PC9 Cells

FIG. 9 shows results of FACs analysis of mAb116 on PC9 cells. PC9 FACS titration of mAb116 was performed by incubating PC9 cells with a serial dilution (30000 pM to 0.1 pM, 3 fold serial dilution) of mAb116 for 30 min on ice, before the cells were stained with Alexa488-conjugated anti-mouse IgG (Jackson lab) for 30 min. MFI was analyzed using BD C6. Affinity K_{D} was determined to be about 980 pM (or 0.98 nM).

FIG. 10 shows that PC9 cells internalized bound mAb116. Live PC cells were cultured on coverslips, and were incubated with 10 μg/mL mAb116 for 1 hr on ice, before the cells were washed 3 times with PBS. Cells were then cultured at 37° C. for 0 hr, 2 hr, or 4 hr, before fixation with 4% PFA before detected with FITC conjugated secondary antibody by FACs. PC9 cells were then co-stained by mAb116 (labeled by a green fluorescent dye Alexa488) and anti-LAMP1 (labeled by a red fluorescent dye Alexa595). Specifically, PC9 cells were permeablized with 0.1% Triton X and incubated with mAb116 and Rabbit anti-LAMP1 antibody (1:200, Abcam) and mAb116 for 1 hr. Antibodies were then labeled with Alexa488 conjugated anti-Mouse antibody and Alexa595 conjugated anti-Rabbit antibody, respectively. Colocalization of mAb116 and anti-LAMP1 signals results in yellow signal that indicates internalization of mAb116 by PC9 cells to the lysosomal compartment. mAb116 was first observed on the cell surface without any co-localization with LAMP1 at 0 hr. Colocalization of mAb116 and LAMP1 was observed at 2 hr and 4 hr.

FACs analysis based on surface fluorescence shows mAb116 internalization on PC9 cells (data not shown). Specifically, live PC9 cells were incubated with 10 μg/mL mAb116 for 0.5 hr on ice before washed 3 times with PBS. Cells were then cultured at 37° C. for 0 hr, 2 hr, or 4 hr, before fixation with 4% PFA. Cells were then stained with Alexa488 conjugated anti-Mouse antibody and analyzed with FACs by calculating surface MFI. Surface MFI, which represented surface localization of mAb116, reduced by about 90% at 4 hr incubation at 37° C. Shown in quantification of the FACs data, expressed as the mean percent internalization±SEM (n=3) in PC9 cells. The vertical axis represents the relative surface florescence (MFI, %). The data shows that mAb116 could bind membrane antigen and be internalized in PC9 cells.

### Example 7 Indirect Cytotoxicity of mAb116 is Antigen Expression-Dependent

FIG. 11 shows indirect cytotoxicity of mAb116 and control IgG. PC9 cells were cultured in 96-well plate at 2000 cells/well confluence overnight. Cells were then treated with serial dilution of mAb116 or IgG together with 2 μg/mL MMAE-conjugated goat anti mouse IgG antibody for 72 hrs. Cell number was then calculated by CCK8 (dojindo). The mAb116 antibody cocktail inhibited PC9 growth with an IC₅₀ of about 30 pM, but IgG cocktail did not have any effect. Shown is representative data derived from PC9 cells, expressed as the mean percent growth inhibition±SEM (n=3).

### Example 8 mAb116 Targets Human CD44 v9 Exon

FIGS. 12A and 12B show that mAb116 targets human CD44 v9 exon. PC9 was transfected with siRNA targeting human CD44 V9 epitope or control siRNA for 48 hrs. Transfected cells were then either stained with mAb116 and analyzed by FACs, or the total protein was extracted and the abundance of mAb116 antigen was evaluated by Western blotting. Knockdown of CD44v9 decreased mAb116 surface staining intensity in FACs (FIG. 12A, FACs data showing that CD44v9 siRNA (V9.si) inhibits surface signal of mAb116 (representative of n=3). Knockdown of CD44v9 also decreased protein expression level of the mAb116 antigen (FIG. 12B). The data suggests that mAb116 targets CD44v9.

An antibody-drug-conjugate was prepared using the mAb116 as the antigen-binding moiety. FIG. 13A shows a schematic drawing of the structure of the mAb116-ADC (AMT116), in which mAb116 was conjugated with MC-vc-PAB-MMAE. HPLC-HIC (Hydrophobic Interaction Chromatography) of AMT116 shows that the average drug-antibody ratio (DAR) was about 4.23. FIG. 13B.

FIG. 14 shows cytotoxicity of AMT116 in PC9 and KYSE-150 (Esophagus Carcinoma cell line) cells. Graphs are representative data derived from PC9 and KYSE-150 cells showing the mean percent growth inhibition±SEM of AMT116 and IgG control (n=3). The IC₅₀ values of AMT116 were 134 pM and 670.2 pM in PC9 and KYSE-150 cells, respectively.

FIG. 15 shows in vivo efficacy of AMT116. About 5×10⁶ KYSE-150 cells were suspended in 1:1 Matrigel before injection into the right flank of female Balb/c nude mice (8-10 weeks, 20-22 g). Tumor volume (measured by 0.5×length×width²) and body weight were determined at least twice weekly. Mice were grouped randomly (n=5/group) based on their initial tumor size (median tumor volume of approximately 250-500 mm³) before dosing. Vehicle (PBS), AMT116, or control ADC was administered by i.v. infusion (3 mg/kg, q3d×3). Group mean (±SEM) tumor volumes were plotted over the duration of study.

### Example 9 Expression of CD44v9 in Human Non-Small-Cell Lung Cancer and Other Cancers

FIGS. 16A and 16B show expression of CD44v9 in human non-small-cell lung cancer (right panels of FIG. 16A) and normal lung tissues (left panel of FIG. 16A). IHC detection of CD44v9 protein using mAb116 antibody is shown from a series of normal and cancer tissues, showing that CD44v9 was up-regulated in a tumor-specific manner. Photomicrograph images depict tumor tissues representative of 0, 1+, 2+ and 3+ staining intensity (right panels of FIG. 16A). FIG. 16B shows prevalence of CD44v9 in different subtypes of NSCLC. SCC, Squamous cell carcinoma; LCC, Large cell carcinoma.

FIG. 17 shows overexpression of CD44v9 in multiple tumor types. IHC detection of CD44v9 protein using mAb116 antibody is shown from a series of normal and cancer tissues, showing that CD44v9 was up-regulated in a tumor-specific manner.

### Example 10 Antibody Sequences

The sequences of the various regions/domains of the mAb119 and mAb116 monocloncal antibodies are listed below.

TABLE 1
Sequence of Variable Region of mAb119

| Chains | Heavy Chain (H) | Light Chain (L) |
|---|---|---|
| FR1-IMGT | EVQLQESGPELKKPGETVKISCKAS | DIQMTQTTSSLSASLGDRVTITCSAS |
| | (SEQ ID NO: 4) | (SEQ ID NO: 13) |
| CDR1-IMGT | GYIFTNYG | QGISNY |
| | (SEQ ID NO: 1) | (SEQ ID NO: 10) |
| FR2-IMGT | MNWVKQAPGKGLKWMGW | LNWYQQKPDGTVKLLIF |
| | (SEQ ID NO: 5) | (SEQ ID NO: 14) |
| CDR2-IMGT | INTYTGEP | YTS |
| | (SEQ ID NO: 2) | (SEQ ID NO: 11) |
| FR3-IMGT | TYADDFKGRFAFSLETSASTAYLQI | TLPSGVPSRFSGSGSGTDYSLTIRNL |
| | NNLKNEDTATYFC | EPEDFATYCC |
| | (SEQ ID NO: 6) | (SEQ ID NO: 15) |
| CDR3-IMGT | ARTDYYGSSYWYFDV | QQYSKFPYT |
| | (SEQ ID NO: 3) | (SEQ ID NO: 12) |
| JUNCTION | CARTDYYGSSYWYFDVW | CQQYSKFPYTF |
| | (SEQ ID NO: 7) | (SEQ ID NO: 16) |
| J-REGION | YWYFDVWGAGTTVTVSS | YTFGGGTKLEIK |
| | (SEQ ID NO: 8) | (SEQ ID NO: 17) |
| FR4-IMGT | WGAGTTVTVSS | FGGGTKLEIK |
| | (SEQ ID NO: 9) | (SEQ ID NO: 18) |

| Antigen peptide | siRNA sequence (sense) |
|---|---|
| HEGYRQTPKE | GGCAACUCCUAGUAGUACATT |
| (SEQ ID NO: 19) | (SEQ ID NO: 20) |
| | GAAGACUCCCAUUCGACAATT |
| | (SEQ ID NO: 21) |
| | GCAACTCCTAGTAGTACAAdTdT |
| | (SEQ ID NO: 22) |
| | TGAGGGATATCGCCAAACAdTdT |
| | (SEQ ID NO: 23) |

HEGYRQTPKE (SEQ ID NO: 19)-CD44v6 epitope sequence used to raise the subject anti-CD44v6 antibody.
HEGYRQTPKEDS (SEQ ID NO: 24)

TABLE 2
Sequence of Variable Region of mAb116

| Chains | Heavy Chain (H) | Light Chain (L) |
|---|---|---|
| FRI-IMGT | QVQLQQSGAELVKPGASVKLSCTAS | DIVMTQSAPSVPVTPGESVSISCRSS |
| | (SEQ ID NO: 28) | (SEQ ID NO: 37) |
| CDR1-IMGT | GFNIKDTY | KSLLHSNGNTY |
| | (SEQ ID NO: 25) | (SEQ ID NO: 34) |
| FR2-IMGT | MHWVKQRPEQGLEWIGR | LYWFLQRPGQSPQLLIH |
| | (SEQ ID NO: 29) | (SEQ ID NO: 38) |
| CDR2-IMGT | IDPANGNT | RMS |
| | (SEQ ID NO: 26) | (SEQ ID NO: 35) |
| FR3-IMGT | KYDPKFQGKATITADTSSNTAYLQL | NLASGVPDRFSGSGSGTAFTLRISRV |
| | SSLTSEDTAVYYC | EAEDVGVYYC |
| | (SEQ ID NO: 30) | (SEQ ID NO: 39) |
| CDR3-IMGT | ASRGEGTGFAY | MQHLEYPFT |
| | (SEQ ID NO: 27) | (SEQ ID NO: 36) |
| JUNCTION | CASRGEGTGFAYW | CMQHLEYPFTF |
| | (SEQ ID NO: 31) | (SEQ ID NO: 40) |
| J-REGION | FAYWGLGTLVTVSA | TFGGGTKLEIK |
| | (SEQ ID NO: 32) | (SEQ ID NO: 41) |
| FR4-IMGT | WGLGTLVTVSA | FGGGTKLEIK |
| | (SEQ ID NO: 33) | (SEQ ID NO: 42) |

SHEGLEEDKD (SEQ ID NO: 43)-CD44v9 epitope sequence used to raise the subject anti-CD44v9 antibody.
SHEGLEEDKDH (SEQ ID NO: 44)

1-9. (canceled)

10. An isolated monoclonal antibody, or an antigen-binding fragment thereof, specific for an isolated CD44v9 epitope, wherein said CD44v9 epitope comprises/consists essentially of SEQ ID NO: 43 (e.g., an epitope consisting of SEQ ID NO: 43 plus 1 or 2 residues on the N-terminus of SEQ ID NO: 43, SEQ ID NO: 19 plus 1 or 2 residues on the C-terminus of SEQ ID NO: 43, or SEQ ID NO: 43 plus 1 or 2 residues on both the N-terminus and the C-terminus of SEQ ID NO: 43), or consists of SEQ ID NO: 43; preferably, said antibody or antigen-binding fragment thereof is raised against said isolated CD44v9 epitope, or raised against a fusion protein or chemical conjugate thereof comprising said isolated CD44v9 epitope and a carrier protein (such as albumin, preferably BSA or ovalbumin, or keyhole limpet hemocyanin (KLH)).

11. The isolated monoclonal antibody or antigen-binding fragment thereof according to claim 10, wherein said monoclonal antibody comprises:
(1) a heavy chain variable region (HCVR), comprising a HCVR CDR1 sequence of SEQ ID NO: 25, a HCVR CDR2 sequence of SEQ ID NO: 26, and a HCVR CDR3 sequence of SEQ ID NO: 27;
(2) a light chain variable region (LCVR), comprising a LCVR CDR1 sequence of SEQ ID NO: 34, a LCVR CDR2 sequence of SEQ ID NO: 35, and a LCVR CDR3 sequence of SEQ ID NO: 36.

12. The isolated monoclonal antibody or antigen-binding fragment thereof according to claim 10, wherein the CD44v9 epitope is SEQ ID NO: 43.

13. The isolated monoclonal antibody or antigen-binding fragment thereof according to claim 10, wherein the CD44v9 epitope consists essentially of SEQ ID NO: 43 (e.g., an epitope consisting of SEQ ID NO: 43 plus 1 or 2 residues on the N-terminus of SEQ ID NO: 43, SEQ ID NO: 43 plus 1 or 2 residues on the C-terminus of SEQ ID NO: 43, or SEQ ID NO: 43 plus 1 or 2 residues on both the N-terminus and the C-terminus of SEQ ID NO: 43).

14. The isolated monoclonal antibody or antigen-binding fragment thereof according to claim 13, wherein the CD44v9 epitope is SEQ ID NO: 44 (SHEGLEEDKDH).

15. The isolated monoclonal antibody or antigen-binding fragment thereof according to claim 11, wherein:
(i) the HCVR further comprises one or more of SEQ ID NOs: 28-33; and/or, (ii) the LCVR further comprises one or more of SEQ ID NOs: 37-42.

16. The isolated monoclonal antibody or antigen-binding fragment thereof according to claim 10, which binds to said CD44v9 epitope, or a cell having said CD44v9 epitope, with a KD of about 10 nM, about 5 nM, about 2 nM, about 1 nM or less.

17. The isolated monoclonal antibody or antigen-binding fragment thereof according to claim 10, which is a human-mouse chimeric antibody, a humanized antibody, a human antibody, a CDR-grafted antibody, or a resurfaced antibody.

18. The isolated monoclonal antibody or antigen-binding fragment thereof according to claim 10, wherein said antigen-binding fragment thereof is an Fab, Fab′, F(ab′)₂, Fd, single chain Fv or scFv, disulfide linked Fv, V-NAR domain, IgNar, intrabody, IgGΔCH2, minibody, F(ab′)3, tetrabody, triabody, diabody, single-domain antibody, DVD-Ig, Fcab, mAb2, (scFv)2, or scFv-Fc.

19-20. (canceled)

21. A polypeptide comprising the HCVR and/or the LCVR of claim 10.

22-23. (canceled)

24. A polynucleotide encoding the polypeptide of claim 21.

25-26. (canceled)

27. A vector comprising the polynucleotide of claim 24.

28-33. (canceled)

34. A cell comprising the antibody or antigen-binding fragment thereof of claim 10.

35-40. (canceled)

41. A method of producing the antibody or antigen-binding fragment thereof of claim 10, comprising:
(a) culturing the cell of claim 34,
(b) isolating said antibody, antigen-binding fragment thereof, or polypeptide from said cultured cell.

42-52. (canceled)

53. An immunoconjugate (or antibody-drug conjugate or ADC) having the following formula:
Ab-[-L-D]n,
wherein:
Ab is an antibody or antigen-binding fragment thereof of claim 10, that is covalently linked to one or more units of linker-drug moieties -[-L-D], wherein L is a linker and D is a cytotoxic drug; and,
n is an integer from 1 to 20 (such as from 1-12); and
wherein each linker-drug moiety may have the same or different linker L or cytotoxic drug D.

54-63. (canceled)

64. A pharmaceutical composition comprising the antibody or antigen-binding fragment thereof of claim 10, and a pharmaceutically acceptable carrier or excipient.

65-68. (canceled)

69. A method for inhibiting the growth of a cell expressing CD44v9, comprising contacting the cell with the antibody or antigen-binding fragment thereof of claim 10.

70-76. (canceled)

77. A method for treating a subject having cancer, wherein cells of the cancer expresses CD44v9, the method comprising administering to said subject a therapeutically effective amount of an antagonist of CD44v9 comprising the CD44v9 antibody or an antigen-binding fragment thereof of claim 10.

78. A method for treating a cell-proliferative disorder in a subject, wherein cells of the cell-proliferative disorder expresses CD44v9, the method comprising administering to said subject a therapeutically effective amount of an antagonist of CD44v9 comprising the CD44v9 antibody or an antigen-binding fragment thereof of claim 10.

79-81. (canceled)

82. A method of determining presence and/or abundance of CD44v9 in a sample from a subject, the method comprising contacting the sample with the antibody or antigen-binding fragment thereof of claim 10.

83. (canceled)

84. A method of diagnosing and treating a subject having cancer, wherein cells of the cancer expresses CD44v9, the method comprising:
(1) determining presence and/or abundance of CD44v9 in a cancer sample from the subject in order to identify subject expressing CD44v9 in the cancer sample;
(2) administering to said subject a therapeutically effective amount of the antibody or antigen-binding fragment thereof of claim 10;
thereby diagnosing and treating the subject having cancer.


SEQUENCE LISTING (45 sequences)

SEQ ID NO: 1 - protein, 8 aa, Artificial Sequence
  Description of Artificial Sequence Synthetic peptide
GYIFTNYG

SEQ ID NO: 2 - protein, 8 aa, Artificial Sequence
  Description of Artificial Sequence Synthetic peptide
INTYTGEP

SEQ ID NO: 3 - protein, 15 aa, Artificial Sequence
  Description of Artificial Sequence Synthetic peptide
ARTDYYGSSYWYFDV

SEQ ID NO: 4 - protein, 25 aa, Artificial Sequence
  Description of Artificial Sequence Synthetic peptide
EVQLQESGPELKKPGETVKISCKAS

SEQ ID NO: 5 - protein, 17 aa, Artificial Sequence
  Description of Artificial Sequence Synthetic peptide
MNWVKQAPGKGLKWMGW

SEQ ID NO: 6 - protein, 38 aa, Artificial Sequence
  Description of Artificial Sequence Synthetic polypeptide
TYADDFKGRFAFSLETSASTAYLQINNLKNEDTATYFC

SEQ ID NO: 7 - protein, 17 aa, Artificial Sequence
  Description of Artificial Sequence Synthetic peptide
CARTDYYGSSYWYFDVW

SEQ ID NO: 8 - protein, 17 aa, Artificial Sequence
  Description of Artificial Sequence Synthetic peptide
YWYFDVWGAGTTVTVSS

SEQ ID NO: 9 - protein, 11 aa, Artificial Sequence
  Description of Artificial Sequence Synthetic peptide
WGAGTTVTVSS

SEQ ID NO: 10 - protein, 6 aa, Artificial Sequence
  Description of Artificial Sequence Synthetic peptide
QGISNY

SEQ ID NO: 11 - protein, 3 aa, Artificial Sequence
  Description of Artificial Sequence Synthetic peptide
YTS

SEQ ID NO: 12 - protein, 9 aa, Artificial Sequence
  Description of Artificial Sequence Synthetic peptide
QQYSKFPYT

SEQ ID NO: 13 - protein, 26 aa, Artificial Sequence
  Description of Artificial Sequence Synthetic peptide
DIQMTQTTSSLSASLGDRVTITCSAS

SEQ ID NO: 14 - protein, 17 aa, Artificial Sequence
  Description of Artificial Sequence Synthetic peptide
LNWYQQKPDGTVKLLIF

SEQ ID NO: 15 - protein, 36 aa, Artificial Sequence
  Description of Artificial Sequence Synthetic polypeptide
TLPSGVPSRFSGSGSGTDYSLTIRNLEPEDFATYCC

SEQ ID NO: 16 - protein, 11 aa, Artificial Sequence
  Description of Artificial Sequence Synthetic peptide
CQQYSKFPYTF

SEQ ID NO: 17 - protein, 12 aa, Artificial Sequence
  Description of Artificial Sequence Synthetic peptide
YTFGGGTKLEIK

SEQ ID NO: 18 - protein, 10 aa, Artificial Sequence
  Description of Artificial Sequence Synthetic peptide
FGGGTKLEIK

SEQ ID NO: 19 - protein, 10 aa, Artificial Sequence
  Description of Artificial Sequence Synthetic peptide
HEGYRQTPKE

SEQ ID NO: 20 - DNA, 21 bp, Artificial Sequence
  Description of Artificial Sequence Synthetic oligonucleotide
  Description of Combined DNA/RNA Molecule Synthetic oligonucleotide
ggcaacuccuaguaguacatt

SEQ ID NO: 21 - DNA, 21 bp, Artificial Sequence
  Description of Artificial Sequence Synthetic oligonucleotide
  Description of Combined DNA/RNA Molecule Synthetic oligonucleotide
gaagacucccauucgacaatt

SEQ ID NO: 22 - DNA, 21 bp, Artificial Sequence
  Description of Artificial Sequence Synthetic oligonucleotide
gcaactcctagtagtacaatt

SEQ ID NO: 23 - DNA, 21 bp, Artificial Sequence
  Description of Artificial Sequence Synthetic oligonucleotide
tgagggatatcgccaaacatt

SEQ ID NO: 24 - protein, 12 aa, Artificial Sequence
  Description of Artificial Sequence Synthetic peptide
HEGYRQTPKEDS

SEQ ID NO: 25 - protein, 8 aa, Artificial Sequence
  Description of Artificial Sequence Synthetic peptide
GFNIKDTY

SEQ ID NO: 26 - protein, 8 aa, Artificial Sequence
  Description of Artificial Sequence Synthetic peptide
IDPANGNT

SEQ ID NO: 27 - protein, 11 aa, Artificial Sequence
  Description of Artificial Sequence Synthetic peptide
ASRGEGTGFAY

SEQ ID NO: 28 - protein, 25 aa, Artificial Sequence
  Description of Artificial Sequence Synthetic peptide
QVQLQQSGAELVKPGASVKLSCTAS

SEQ ID NO: 29 - protein, 17 aa, Artificial Sequence
  Description of Artificial Sequence Synthetic peptide
MHWVKQRPEQGLEWIGR

SEQ ID NO: 30 - protein, 38 aa, Artificial Sequence
  Description of Artificial Sequence Synthetic polypeptide
KYDPKFQGKATITADTSSNTAYLQLSSLTSEDTAVYYC

SEQ ID NO: 31 - protein, 13 aa, Artificial Sequence
  Description of Artificial Sequence Synthetic peptide
CASRGEGTGFAYW

SEQ ID NO: 32 - protein, 14 aa, Artificial Sequence
  Description of Artificial Sequence Synthetic peptide
FAYWGLGTLVTVSA

SEQ ID NO: 33 - protein, 11 aa, Artificial Sequence
  Description of Artificial Sequence Synthetic peptide
WGLGTLVTVSA

SEQ ID NO: 34 - protein, 11 aa, Artificial Sequence
  Description of Artificial Sequence Synthetic peptide
KSLLHSNGNTY

SEQ ID NO: 35 - protein, 3 aa, Artificial Sequence
  Description of Artificial Sequence Synthetic peptide
RMS

SEQ ID NO: 36 - protein, 9 aa, Artificial Sequence
  Description of Artificial Sequence Synthetic peptide
MQHLEYPFT

SEQ ID NO: 37 - protein, 26 aa, Artificial Sequence
  Description of Artificial Sequence Synthetic peptide
DIVMTQSAPSVPVTPGESVSISCRSS

SEQ ID NO: 38 - protein, 17 aa, Artificial Sequence
  Description of Artificial Sequence Synthetic peptide
LYWFLQRPGQSPQLLIH

SEQ ID NO: 39 - protein, 36 aa, Artificial Sequence
  Description of Artificial Sequence Synthetic polypeptide
NLASGVPDRFSGSGSGTAFTLRISRVEAEDVGVYYC

SEQ ID NO: 40 - protein, 11 aa, Artificial Sequence
  Description of Artificial Sequence Synthetic peptide
CMQHLEYPFTF

SEQ ID NO: 41 - protein, 11 aa, Artificial Sequence
  Description of Artificial Sequence Synthetic peptide
TFGGGTKLEIK

SEQ ID NO: 42 - protein, 10 aa, Artificial Sequence
  Description of Artificial Sequence Synthetic peptide
FGGGTKLEIK

SEQ ID NO: 43 - protein, 10 aa, Artificial Sequence
  Description of Artificial Sequence Synthetic peptide
SHEGLEEDKD

SEQ ID NO: 44 - protein, 11 aa, Artificial Sequence
  Description of Artificial Sequence Synthetic peptide
SHEGLEEDKDH

SEQ ID NO: 45 - protein, 8 aa, Artificial Sequence
  Description of Artificial Sequence Synthetic peptide
WSHPQFEK